(19) United States
(12) Patent Application Publication
(10) Pub. No.: US 2023/0235355 A1
(43) Pub. Date: Jul. 27, 2023

(54) MONO- AND MULTI-VALENT SARS-COV- 2 ADENOVIRAL VECTOR VACCINES AND SARS-COV-2 IMMUNE GLOBULIN AND METHODS OF USE

(71) Applicant: EpicentRx, Inc., La Jolla, CA (US)

(72) Inventors: Tony R. REID, San Diego, CA (US); Bryan T. ORONSKY, Los Altos Hills, CA (US)

(73) Assignee: EpicentRx, Inc., La Jolla, CA (US)

(21) Appl. No.: 18/000,815

(22) PCT Filed: Jun. 5, 2021
(86) PCT No.: PCT/US2021/036054

§ 371 Date: Dec. 5, 2022

Related U.S. Application Data
(60) Provisional application No. 63/035,593, filed on Jun. 5, 2020.

(51) Int. Cl. C12N 15/86; C07K 14/165; A61K 9/00; C07K 16/10; A61K 39/12; A61P 31/14; A61P 35/00

(52) U.S. Cl. CPC C12N 15/86; C07K 14/165; A61K 9/0019; C07K 16/10; A61K 39/12; A61P 31/14; A61P 35/00; A61K 2039/5256

(57) ABSTRACT

The present disclosure relates to recombinant human adenoviruses engineered to express a structural protein of a coronavirus. The recombinant adenoviruses are suitable for active immunization against a coronavirus in a human subject. Additionally, immune globulin obtained from immunized human subjects is suitable for passive immunization of a coronavirus-infected human subject.

### CROSS-REFERENCE TO RELATED APPLICATIONS

This application claims priority to and the benefit of U.S. Provisional Application No. 63/035,593, filed Jun. 5, 2020, the disclosure of which is incorporated by reference in its entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name 203592001940SEQLIST.TXT, date recorded: Jun. 3, 2021, size: 95 KB).

### FIELD

The present disclosure relates to recombinant human adenoviruses engineered to express a structural protein of a coronavirus. The recombinant adenoviruses are suitable for active immunization against a coronavirus in a human subject. Additionally, immune globulin obtained from immunized human subjects is suitable for passive immunization of a coronavirus-infected human subject.

### BACKGROUND

Coronavirus disease 2019 (COVID-19) is a highly infectious disease caused by severe acute respiratory syndrome coronavirus 2 (SARS-CoV-2). Although the majority of infected individuals experience only mild symptoms, some infected individuals develop acute respiratory distress syndrome, multi-organ failure, septic shock and blood clots. As such there is significant morbidity and mortality associated with COVID-19.

Currently, there are no vaccines or specific antiviral treatments for COVID-19 that are licensed for use in human subjects. Thus, there is an urgent global need for prophylactic vaccines and therapeutic biologic products to confront the COVID-19 pandemic.

### BRIEF SUMMARY

The present disclosure relates to recombinant human adenoviruses engineered to express a structural protein of a coronavirus. The recombinant adenoviruses are suitable for active immunization against a coronavirus in a human subject. Additionally, immune globulin obtained from immunized human subjects is suitable for passive immunization of a coronavirus-infected human subject.

### BRIEF DESCRIPTION OF THE DRAWINGS

FIG. 1 is a schematic of the nVAX-19 genome. nVAX-19 is a recombinant human adenovirus type 5 engineered to express the SARS-CoV-2 nucleocapsid (N) protein. In the schematic, early transcription units are shown above and late transcription units are shown below the line representing the linear double stranded DNA genome. Modifications in nVAX-19 include the TAV-255 deletion in the E1A promoter and replacement of a portion of the E1B-19K gene with cDNA encoding SARS-CoV-2 N.

FIG. 2 is a schematic of the sVAX-19 genome. sVAX-19 is a recombinant human adenovirus type 5 engineered to express the SARS-CoV-2 spike (S) protein. In the schematic, early transcription units are shown above and late transcription units are shown below the line representing the linear double stranded DNA genome. Modifications in sVAX-19 include the TAV-255 deletion in the E1A promoter, insertion of an expression cassette between the L5 and E4 transcription units to drive expression of SARS-CoV-2 S, and deletions of E3 and part of E4.

FIG. 3 is a schematic of the Geist-20 genome. Geist-20 is a recombinant human adenovirus type 5 engineered to express the SARS-CoV-2 spike (S), membrane (M) and envelope (E) proteins. In the schematic, early transcription units are shown above and late transcription units are shown below the line representing the linear double stranded DNA genome. Modifications in Geist-20 include the TAV-255 deletion in the E1A promoter, insertion of an expression cassette between the L5 and E4 transcription units to drive expression of SARS-CoV-2 S, deletions of E3 and part of E4, insertion of an expression cassette between the transcription units for IX and E2B to drive expression of SARS-CoV-2 M and E.

### DETAILED DESCRIPTION

The present disclosure relates to recombinant human adenoviruses engineered to express a structural protein of a coronavirus. The recombinant adenoviruses are suitable for active immunization against a coronavirus in a human subject. Additionally, immune globulin obtained from immunized human subjects is suitable for passive immunization of a coronavirus-infected human subject.

### General Techniques and Definitions

The practice of the present disclosure will employ, unless otherwise indicated, conventional techniques of molecular biology (including recombinant techniques), cell biology, biochemistry, virology and immunology, which are understood by one of ordinary skill in the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

At various places in the present specification, viruses, compositions, systems, processes and methods, or features thereof, are disclosed in groups or in ranges. It is specifically intended that the description include each and every individual subcombination of the members of such groups and ranges. By way of example, an integer in the range of 1 to 20 is specifically intended to individually disclose 1, 2, 3, 4, 5, 6, 7, 8, 9, 10, 11, 12, 13, 14, 15, 16, 17, 18, 19 and 20.

It should be understood that the order of steps or order for performing certain actions is immaterial so long as the present disclosure remains operable. Moreover, two or more steps or actions may be conducted simultaneously.

The use of any and all examples, or exemplary language herein, for example, “such as” or “including,” is intended merely to illustrate better the present invention and does not pose a limitation on the scope of the disclosure unless claimed. No language in the specification should be construed as indicating any non-claimed element as essential to the practice of the present disclosure.

The term “about” as used herein in reference to a value, encompasses from 90% to 110% of that value (e.g., a recombinant adenovirus dose of about 20 mg/kg refers to a dose of 18 mg/kg to 22 mg/kg).

An “effective amount” or a “sufficient amount” of a substance is that amount sufficient to effect beneficial or desired results, including clinical results, and, as such, an “effective amount” depends upon the context in which it is being applied. In the context of administering a recombinant human adenovirus engineered to express a coronavirus structural protein, an effective amount contains sufficient recombinant adenovirus to stimulate an immune response against the coronavirus structural protein (preferably a seroprotective level of coronavirus-neutralizing antibody).

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats).

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is naturally associated (e.g., removed from its original environment). The term “isolated,” when used in reference to a coronavirus immune globulin refers to immune globulin that has been removed from plasma of a subject in which a coronavirus-neutralizing antibody response has been elicited.

“Stimulation” of a response or parameter includes eliciting and/or enhancing that response or parameter when compared to otherwise same conditions except for a parameter of interest, or alternatively, as compared to another condition (e.g., increase in coronavirus-reactive antibodies after administration of a coronavirus vaccine to a study subject as compared to administration of a placebo to a control subject). For example, “stimulation” of an immune response means an increase in the response. Depending upon the parameter measured, the increase may be from 5-fold to 500-fold or over, or from 5, 10, 50, or 100-fold to 500, 1,000, 5,000, or 10,000-fold.

“Inhibition” of a response or parameter includes blocking and/or suppressing that response or parameter when compared to otherwise same conditions except for a parameter of interest, or alternatively, as compared to another condition (e.g., decrease in coronavirus-associated symptoms after administration of a coronavirus vaccine to a study subject as compared to administration of a placebo to a control subject). For example, “inhibition” of an immune response means a decrease in the response.

The terms “treating” or “treatment” of a disease refer to executing a protocol, which may include administering one or more drugs to an individual (human or otherwise), in an effort to alleviate a sign or symptom of the disease. Thus, “treating” or “treatment” does not require complete alleviation of signs or symptoms, does not require a cure, and specifically includes protocols that have only a palliative effect on the individual. As used herein, and as well-understood in the art, “treatment” is an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival of an individual not receiving treatment. “Palliating” a disease or disorder means that the extent and/or undesirable clinical manifestations of the disease or disorder are lessened and/or time course of progression of the disease or disorder is slowed, as compared to the expected untreated outcome. Further, palliation and treatment do not necessarily occur by administration of one dose, but often occur upon administration of a series of doses.

As used herein the term “immunization” refers to a process that increases a subject's reaction to antigen and therefore improves the ability of the subject to resist or overcome infection.

The term “vaccination” as used herein refers to the introduction of vaccine into a body of a subject.

### I. Recombinant Adenoviral Vectors

A recombinantly modified virus is referred to herein as a “recombinant virus.” A recombinant virus may be modified by recombinant DNA techniques to be replication deficient, conditionally replicating, or replication competent, and/or to express a heterologous coding region (e.g., exogenous transgene) of a viral pathogen, such as a structural protein of a human coronavirus. The recombinant virus of the present disclosure is a recombinant adenovirus.

Adenoviruses are medium-sized (90-100 nm), non-enveloped (naked), icosahedral viruses composed of a nucleocapsid and a double-stranded linear DNA genome. Adenoviruses replicate in the nucleus of mammalian cells using the host's replication machinery. The term “adenovirus” refers to any virus in the genus Adenoviridiae including, but not limited to, human, bovine, ovine, equine, canine, porcine, murine, and simian adenovirus subgenera. In particular, human adenoviruses include the A-F subgenera, as well as the individual serotypes thereof. The individual serotypes and A-F subgenera including but are not limited to human adenovirus types 1, 2, 3, 4, 4a, 5, 6, 7, 8, 9, 10, 11 (Ad11a and Ad11p), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. Preferred recombinant adenoviruses are derived from human adenovirus types 5 (Ad5). Unless stated otherwise, all Ad5 nucleotide numbers are relative to the NCBI No. AC_000008.1, the nucleotide sequence of which is set forth as SEQ ID NO:1 and incorporated by reference.

The adenovirus replication cycle has two phases: an early phase, during which four transcription units E1, E2, E3, and E4 are expressed, and a late phase which occurs after the onset of viral DNA synthesis when late transcripts are expressed primarily from the major late promoter (MLP). The late messages encode most of the virus's structural proteins. The gene products of E1, E2 and E4 are responsible for transcriptional activation, cell transformation, viral DNA replication, as well as other viral functions, and are necessary for viral growth.

The E1a gene of Ad5 is processed by mRNA splicing to yield five distinct isoforms; 13S, 12S, 11S, 10S and 9S. The major forms 13S and 12S code for two E1a proteins, 289R and 243R respectively, that regulate transcription of both viral and cellular genes in adenovirus-infected cells and are essential for adenoviral replication. The 289R form includes a critical transactivation domain that activates transcription of the early adenoviral genes: E2, E3, and E4. This domain is spliced out to generate the 243R isoform of E1a and viruses expressing only the 243R form are unable to transactivate expression from the early viral genes. E1a induces expression of cellular genes including c-Fos, c-Jun, and c-Myc and represses the transcription of c-erbB2 and epidermal growth factor receptor. E1a proteins can drive quiescent cells into cell division by interaction with critical cellular cell cycle proteins including pRB, p27, cyclin A, cyclin E, CtBP, and p300/CBP.

The general structure of the mature Adenovirion is conserved among different Adenoviral species. The Adenoviral capsid is composed of three major proteins (II, III, and IV) and five minor proteins, VI, VIII, IX, IIIa, and IVa2. “IVa2 gene” used herein refers to the gene encoding the IVa2 protein, modified versions, and/or fragment thereof. “IX gene” used herein refers to the gene encoding the IX protein, modified versions, and/or fragment thereof.

Primary transcripts from E4 are subject to alternative splicing events and are predicted to encode seven different polypeptides: ORF1, ORF2, ORF3, ORF3/4, ORF4, ORF5, ORF6, and ORF6/7. “ORF” is used herein to refer to either the polypeptide or the nucleotide sequence encoding the polypeptide, modified versions, and/or fragment thereof.

In addition, the fiber protein (also known as protein IV or SPIKE) forms spikes that protrude from each vertex of the icosahedral capsid. “Fiber gene” used herein refers to the gene encoding the fiber protein, also known as L5 gene, modified versions, and/or fragment thereof.

A. Modified E1a Transcriptional Control Region

In certain embodiments, the recombinant adenoviruses comprise one or more modifications to a regulatory sequence or promoter. A modification to a regulatory sequence or promoter comprises a deletion, substitution, or addition of one or more nucleotides compared to the wild-type sequence of the regulatory sequence or promoter.

In one embodiment, the modification of a regulatory sequence or promoter comprises a modification of sequence of a transcription factor binding site to reduce affinity for the transcription factor, for example, by deleting a portion thereof, or by inserting a single point mutation into the binding site. In certain embodiments, the additional modified regulatory sequence enhances expression in neoplastic cells, but attenuates expression in normal cells.

In one embodiment, at least one of these seven binding sites, or a functional binding site, is deleted. As used herein, a “functional binding site” refers to a binding site that is capable of binding to a respective binding partner, e.g., a transcription factor, e.g., a binding site that has at least 100%, at least 90%, at least 80%, at least 70%, at least 60%, at least 50%, or at least 40%, of the binding activity of a corresponding wild-type binding site sequence. As used herein, a “non-functional binding site” refers to a binding site that, e.g., has less than 30%, less than 20%, less than 10%, or 0% of the binding activity of a corresponding wild-type binding site sequence.

In certain embodiments, the recombinant adenoviruses comprise a modified E1a regulatory sequence. In certain embodiments, a disclosed recombinant adenovirus may, e.g., comprise a deletion of a functional E1a coding region. As used herein, a “functional E1a coding region” refers to an E1a coding region that encodes for a functional E1a protein, e.g., an E1a protein that is capable of binding to a respective binding partner (e.g., CREB binding protein (CBP)), e.g., an E1a protein that has at least 100%, at least 90%, at least 80%, at least 70%, at least 60%, at least 50%, or at least 40%, of the binding activity of a corresponding wild-type E1a protein. As used herein, a “non-functional E1a coding region” refers to a coding region that encodes for an E1a protein that, e.g., has less than 30%, less than 20%, less than 10%, or 0% of the binding activity of a corresponding wild-type E1a protein.

In certain embodiments, the deletion of a functional E1a coding region comprises a deletion of nucleotides corresponding to the entire coding region of the E1a gene. In certain embodiments, the deletion of a functional E1a coding region comprises a deletion of nucleotides corresponding to 560-1545 of the Ad5 genome or a deletion of nucleotides corresponding to 557-1678 of the Ad5 genome (SEQ ID NO:1). In certain embodiments, the deletion of a functional E1a coding region results in a recombinant adenovirus comprising the sequence GACTGTGCGC (SEQ ID NO:3). In certain embodiments, the recombinant adenovirus includes an E1a insertion site, e.g., the adenovirus has a transgene inserted into the deletion of a functional E1a coding region.

The adenoviral E1b-19k gene functions primarily as an anti-apoptotic gene and is a homolog of the cellular anti-apoptotic gene, BCL-2. Since host cell death prior to maturation of the progeny viral particles would restrict viral replication, E1b-19k is expressed as part of the E1 cassette to prevent premature cell death thereby allowing the infection to proceed and yield mature virions. Accordingly, in certain embodiments, a recombinant adenovirus is provided that includes a deletion of a functional E1b-19k coding region.

A disclosed recombinant adenovirus may, e.g., comprise a deletion of a functional E1b-19k coding region. As used herein, a “functional E1b-19k coding region” refers to an E1b-19k coding region that encodes for a functional E1b-19k protein, e.g., an E1b-19k protein that is capable of binding to a respective binding partner (e.g., BAK), e.g., an E1b-19k protein that has at least 100%, at least 90%, at least 80%, at least 70%, at least 60%, at least 50%, or at least 40%, of the binding activity of a corresponding wild-type E1b-19k protein. As used herein, a “non-functional E1b-19k coding region” refers to a coding region that encodes for an E1b-19k protein that, e.g., has less than 30%, less than 20%, less than 10%, or 0% of the binding activity of a corresponding wild-type E1b-19k protein.

In certain embodiments, the deletion of a functional E1b-19k coding region comprises a deletion located between the start site of E1b-19K (i.e., the nucleotide sequence encoding the start codon of E1b-19k, e.g., corresponding to nucleotides 1714-1716 of SEQ ID NO:1) and the start site of E1b-55K (i.e., the nucleotide sequence encoding the start codon of E1b-55k, e.g., corresponding to nucleotides 2019-2021 of SEQ ID NO:1). In certain embodiments, the deletion of a functional E1b-19k coding region comprises a deletion located between the start site of E1b-19K (i.e., the nucleotide sequence encoding the start codon of E1b-19k, e.g., corresponding to nucleotides 1714-1716 of SEQ ID NO:1) and the stop site of E1b-19K (i.e., the nucleotide sequence encoding the stop codon of E1b-19k, e.g., corresponding to nucleotides 2242-2244 of SEQ ID NO:1). Throughout the description and claims, an insertion between two sites, for example, an insertion between (i) a start site of a first gene (e.g., E1b-19k) and a start site of a second gene, (e.g., E1b-55K), (ii) a start site of a first gene and a stop site of a second gene, (iii) a stop site of a first gene and start site of a second gene, or (iv) a stop site of first gene and a stop site of a second gene, is understood to mean that all or a portion of the nucleotides constituting a given start site or a stop site surrounding the insertion may be present or absent in the final virus. Similarly, an insertion between two nucleotides is understood to mean that the nucleotides surrounding the insertion may be present or absent in the final virus.

The term “transgene” refers to an exogenous gene, or fragment thereof, or exogenous polynucleotide sequence. As used herein “transgene” is understood to encompass a single a exogenous gene, or fragment thereof, or exogenous polynucleotide sequence, or multiple (e.g., 1, 2, 3, 4 or 5 or more) exogenous genes, or fragments thereof, or exogenous polynucleotide sequences.

In certain embodiments, the deletion of a functional E1b-19k coding region comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent to the start site of E1b-19K (i.e., the nucleotide sequence encoding the start codon of E1b-19k, e.g., corresponding to nucleotides 1714-1716 of SEQ ID NO:1). In certain embodiments, the deletion of a functional E1b-19k coding region comprises a deletion of about 200 nucleotides, e.g., 203 nucleotides adjacent to the start site of E1b-19K. In certain embodiments, the deletion of a functional E1b-19k coding region comprises a deletion corresponding to nucleotides 1714-1916 of the Ad5 genome (SEQ ID NO:1). Throughout the description and claims, a deletion adjacent to a site, for example, a deletion adjacent to a start site of a gene or a deletion adjacent to a stop site of a gene, is understood to mean that the deletion may include a deletion of all, a portion, or none of the nucleotides constituting a given start site or a stop site.

In certain embodiments, the recombinant adenovirus comprises one or more exogenous nucleotide sequences inserted in one or more of an E1b-19K insertion site, an E3 insertion site, an E4 insertion site, an IX-E2 insertion site, an L5-E4 insertion site, and any combinations thereof.

In certain embodiments, the recombinant adenovirus comprises an E1b-19K insertion site, e.g., the adenovirus has an exogenous nucleotide sequence inserted into the deletion of a functional E1b-19k coding region. For example, in certain embodiments, an exogenous nucleotide sequence is inserted between nucleotides corresponding to 1714 and 1916 of the Ad5 genome (SEQ ID NO:1). In certain embodiments, an exogenous nucleotide sequence is inserted between CTGACCTC (SEQ ID NO: 4) and TCACCAGG (SEQ ID NO: 5), e.g., the recombinant adenovirus comprises, in a 5′ to 3′ orientation, CTGACCTC (SEQ ID NO: 4), the exogenous nucleotide sequence, and TCACCAGG (SEQ ID NO: 5). CTGACCTC (SEQ ID NO: 4) and TCACCAGG (SEQ ID NO: 5) define unique boundary sequences for a E1b-19K insertion site within the Ad5 genome (SEQ ID NO:1).

In certain embodiments the recombinant adenovirus comprises an E3 deletion. In certain embodiments, the E3 deletion comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides.

In certain embodiments, the E3 deletion comprises a deletion located between the stop site of pVIII (i.e., the nucleotide sequence encoding the stop codon of pVIII, e.g., corresponding to nucleotides 27855-27857 of SEQ ID NO:1) and the start site of Fiber (i.e., the nucleotide sequence encoding the start codon of Fiber, e.g., corresponding to nucleotides 31042-31044 of SEQ ID NO: 1). In certain embodiments, the E3 deletion comprises a deletion located between the stop site of E3-10.5K (i.e., the nucleotide sequence encoding the stop codon of E3-10.5K, e.g., corresponding to nucleotides 29770-29772 of SEQ ID NO:1) and the stop site of E3-14.7K (i.e., the nucleotide sequence encoding the stop codon of E3-14.7K, e.g., corresponding to nucleotides 30837-30839 of SEQ ID NO:1). In certain embodiments, the E3 deletion comprises a deletion of from about 500 to about 1551, from about 500 to about 1500, from about 500 to about 1000, from about 1000 to about 1551, from about 1000 to about 1500, or from about 1500 to about 1551 nucleotides adjacent to the stop site of E3-10.5K. In certain embodiments, the E3 deletion comprises a deletion of about 1050 nucleotides adjacent to the stop site of E3-10.5K (i.e., the nucleotide sequence encoding the stop codon of E3-10.5K, e.g., corresponding to nucleotides 29770-29772 of SEQ ID NO:1), e.g., the E3 deletion comprises a deletion of 1064 nucleotides adjacent to the stop site of E3-10.5K. In certain embodiments, the E3 deletion comprises a deletion corresponding to the Ad5 d1309 E3 deletion. In certain embodiments, the E3 deletion comprises a deletion corresponding to nucleotides 29773-30836 of the Ad5 genome (SEQ ID NO:1).

In certain embodiments, the E3 deletion comprises a deletion located between the stop site of E3-gp19K (i.e., the nucleotide sequence encoding the stop codon of E3-gp19K, e.g., corresponding to nucleotides 29215-29217 of SEQ ID NO:1) and the stop site of E3-14.7K (i.e., the nucleotide sequence encoding the stop codon of E3-14.7K, e.g., corresponding to nucleotides 30837-30839 of SEQ ID NO:1). In certain embodiments, the E3 deletion comprises a deletion of from about 500 to about 1824, from about 500 to about 1500, from about 500 to about 1000, from about 1000 to about 1824, from about 1000 to about 1500, or from about 1500 to about 1824 nucleotides adjacent the stop site of E3-gp19K. In certain embodiments, the E3 deletion comprises a deletion of about 1600 nucleotides adjacent the stop site of E3-gp19K. e.g., the E3 insertion site comprises a deletion of 1622 nucleotides adjacent the stop site of E3-gp19K. In certain embodiments, the E3 deletion comprises a deletion corresponding to nucleotides 29218-30839 of the Ad5 genome (SEQ ID NO:1).

In certain embodiments, the recombinant adenovirus comprises an E3 insertion site, e.g., the adenovirus has an exogenous nucleotide sequence inserted into the E3 deletion. For example, in certain embodiments, an exogenous nucleotide sequence is inserted between nucleotides corresponding to 29773 and 30836 of the Ad5 genome (SEQ ID NO:1). In certain embodiments, the exogenous nucleotide sequence is inserted between CAGTATGA (SEQ ID NO:8) and TAATAAAAAA (SEQ ID NO:9), e.g., the recombinant adenovirus comprises, in a 5′ to 3′ orientation, CAGTATGA (SEQ ID NO:8), the exogenous nucleotide sequence, and TAATAAAAAA (SEQ ID NO:9). CAGTATGA (SEQ ID NO:8) and TAATAAAAAA (SEQ ID NO:9) define unique boundary sequences for an E3 insertion site within the Ad5 genome (SEQ ID NO:1).

In certain embodiments, the exogenous nucleotide sequence is inserted between nucleotides corresponding to 29218 and 30839 of the Ad5 genome (SEQ ID NO:1). In certain embodiments, the exogenous nucleotide sequence is inserted between TGCCTTAA (SEQ ID NO:11) and TAAAAAAAAAT (SEQ ID NO:12), e.g., the recombinant adenovirus comprises, in a 5′ to 3′ orientation, TGCCTTAA (SEQ ID NO:11), the exogenous nucleotide sequence, and TAAAAAAAAAT (SEQ ID NO:12). TGCCTTAA (SEQ ID NO:11) and TAAAAAAAAAT (SEQ ID NO:12) define unique boundary sequences for an E3 insertion site within the Ad5 genome (SEQ ID NO:1).

In certain embodiments, the recombinant adenovirus comprises an E4 deletion. In certain embodiments, the E4 deletion is located between the start site of E4-ORF6/7 (i.e., the nucleotide sequence encoding the start codon of E4-ORF6/7, e.g., corresponding to nucleotides 34075-34077 of SEQ ID NO:1) and the right inverted terminal repeat (ITR; e.g., corresponding to nucleotides 35836-35938 of SEQ ID NO:1). In certain embodiments, the E4 deletion is located between the start site of E4-ORF6/7 and the start site of E4-ORF1 (i.e., the nucleotide sequence encoding the start codon of E4-ORF1, e.g., corresponding to nucleotides 35524-35526 of SEQ ID NO:1). In certain embodiments, the E4 deletion comprises a deletion of a nucleotide sequence between the start site of E4-ORF6/7 and the start site of E4-ORF1. In certain embodiments, the E4 deletion comprises a deletion of from about 500 to about 2500, from about 500 to about 2000, from about 500 to about 1500, from about 500 to about 1000, from about 1000 to about 2500, from about 1000 to about 2000, from about 1000 to about 1500, from about 1500 to about 2500, from about 1500 to about 2000, or from about 2000 to about 2500 nucleotides. In certain embodiments, the E4 deletion comprises a deletion of from about 250 to about 1500, from about 250 to about 1250, from about 250 to about 1000, from about 250 to about 750, from about 250 to about 500, from 500 to about 1500, from about 500 to about 1250, from about 500 to about 1000, from about 500 to about 750, from 750 to about 1500, from about 750 to about 1250, from about 750 to about 1000, from about 1000 to about 1500, or from about 1000 to about 1250 nucleotides adjacent the start site of E4-ORF6/7. In certain embodiments, the E4 deletion comprises a deletion of about 1450 nucleotides adjacent the start site of E4-ORF6/7, e.g., the E4 deletion comprises a deletion of about 1449 nucleotides adjacent the start site of E4-ORF6/7. In certain embodiments, the E4 deletion comprises a deletion corresponding to nucleotides 34078-35526 of the Ad5 genome (SEQ ID NO:1).

In certain embodiments, an E4 insertion site comprises any one of the ORF of the E4 gene. For example, a nucleotide sequence can be inserted in E4 ORF1, and/or E4 ORF2. In certain embodiments, portions of or the entire E4 region may be deleted.

In certain embodiments, the insertion site is the IX-E2 insertion site. In certain embodiments, the IX-E2 insertion site is located between the stop codon of adenovirus IX gene and the stop codon of adenovirus IVa2 gene. In certain embodiments, the exogenous nucleotide sequence is inserted between nucleotides corresponding to 4029 and 4093 of the Ad5 genome (SEQ ID NO:1). In certain embodiments, the exogenous nucleotide sequence is inserted between nucleotides corresponding to 4029 and 4050, nucleotides corresponding to 4051 and 4070, or nucleotides corresponding to 4071 and 4093 of the Ad5 genome (SEQ ID NO:1). In some embodiments, the IX-E2 insertion site has at least 60%, at least 70%, at least 80%, at least 90%, at least 95%, or at least 99% identity to nucleotides corresponding to 4029 and 4093 of the Ad5 genome (SEQ ID NO:1).

In certain embodiments, the insertion site is an L5-E4 insertion site. In certain embodiments, the L5-E4 insertion site is located between the stop codon of adenovirus fiber gene and the stop codon of ORF6 or ORF6/7 of the adenovirus E4 gene. In certain embodiments, the exogenous nucleotide sequence is inserted between nucleotides corresponding to 32785 to 32916 of the Ad5 genome (SEQ ID NO:1). In certain embodiments, the exogenous nucleotide sequence is inserted between nucleotides corresponding to 32785 and 32800, nucleotides corresponding to 32801 and 32820, nucleotides corresponding to 32821 and 32840, nucleotides corresponding to 32841 and 32860, nucleotides corresponding to 32861 and 32880, nucleotides corresponding to 32881 and 32900, or nucleotides corresponding to 32901 and 32916 of the Ad5 genome (SEQ ID NO:1). In some embodiments, the L5-E4 insertion site has at least 60%, at least 70%, at least 80%, at least 90%, at least 95%, or at least 99% identity to nucleotides corresponding to 32785 to 32916 of the Ad5 genome (SEQ ID NO:1).

In certain embodiments, the IX-E2 insertion site comprises a deletion of about 5, 10, 15, 20, 25, 30, 35, 40, 45, 50, 55, or 60 nucleotides. In certain embodiments, the L5-E4 insertion site comprises a deletion of about 5, 10, 15, 20, 25, 30, 35, 40, 45, 50, 55, 60, 65, 70, 75, 80, 85, 90, 95, 100, 105, 110, 115, 120, 125, or 130 nucleotides.

The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

In certain embodiments, the virus has one or more modifications to a regulatory sequence or promoter. A modification to a regulatory sequence or promoter comprises a deletion, substitution, or addition of one or more nucleotides compared to the wild-type sequence of the regulatory sequence or promoter.

In certain embodiments, the modification of a regulatory sequence or promoter comprises a modification of the sequence of a transcription factor binding site to reduce affinity for the transcription factor, for example, by deleting a portion thereof, or by inserting a single point mutation into the binding site. In certain embodiments, the modified regulatory sequence attenuates expression in normal cells.

In certain embodiments, the modified regulatory sequence is operably linked to a sequence encoding a protein. In certain embodiments, at least one of the adenoviral E1a and E1b genes (coding regions) is operably linked to a modified regulatory sequence. In certain embodiments, the E1a gene is operably linked to a modified regulatory sequence.

The E1a regulatory sequence contains five binding sites for the transcription factor Pea3, designated Pea3 I, Pea3 II, Pea3 III, Pea3 IV, and Pea3 V, where Pea3 I is the Pea3 binding site most proximal to the E1a start site, and Pea3 V is most distal. The E1a regulatory sequence also contains binding sites for the transcription factor E2F, hereby designated E2F I and E2F II, where E2F I is the E2F binding site most proximal to the E1a start site, and E2F II is more distal. From the E1a start site, the binding sites are arranged: Pea3 I, E2F I, Pea3 II, E2F II, Pea3 III, Pea3 IV, and Pea3 V.

In certain embodiments, at least one of these seven binding sites, or at least one of seven functional binding sites, is deleted. As used herein, a “functional binding site” refers to a binding site that is capable of binding to a respective binding partner, e.g., a transcription factor, e.g., a binding site that has at least 100%, at least 90%, at least 80%, at least 70%, at least 60%, at least 50%, or at least 40%, of the binding activity of a corresponding wild-type binding site sequence. As used herein, a “non-functional binding site” refers to a binding site that, e.g., has less than 30%, less than 20%, less than 10%, or 0% of the binding activity of a corresponding wild-type binding site sequence.

In certain embodiments, the recombinant adenovirus comprises an E1a promoter having a deletion of a functional Pea3 binding site, e.g., the deletion of an entire Pea3 binding site. As used herein, a “functional Pea3 binding site” refers to a Pea3 binding site that is capable of binding to its respective transcription factor (e.g., Pea3), e.g., a Pea3 binding site that has at least 100%, at least 90%, at least 80%, at least 70%, at least 60%, at least 50%, or at least 40%, of the Pea3 binding activity of a corresponding wild-type Pea3 binding site sequence. As used herein, a “non-functional Pea3 binding site” refers to a Pea3 binding site that, e.g., has less than 30%, less than 20%, less than 10%, or 0% of the Pea3 binding activity of a corresponding wild-type Pea3 binding site sequence. Assays for determining whether a Pea3 binding site binds to Pea3 are known in the art. Exemplary binding assays include electrophoretic mobility shift assays, chromatin immunoprecipitation assays, and DNAse footprinting assays.

In certain embodiments, at least one Pea3 binding site, or a functional Pea3 binding site, is deleted. The deleted Pea3 binding site can be Pea3 I, Pea3 II, Pea3 III, Pea3 IV, and/or Pea3 V. In certain embodiments, the deleted Pea3 binding site is Pea3 II, Pea3 III, Pea3 IV, and/or Pea3 V. In certain embodiments, the deleted Pea3 binding site is Pea3 IV and/or Pea3 V. In another embodiment, the deleted Pea3 binding site is Pea3 II and/or Pea3 III. In certain embodiments, the deleted Pea3 binding site is both Pea3 II and Pea3 III. In certain embodiments, the Pea3 I binding site, or a functional Pea3 I binding site, is retained.

In certain embodiments, at least one E2F binding site, or a functional E2F binding site, is deleted. In certain embodiments, at least one E2F binding site, or a functional E2F binding site, is retained. In certain embodiments, the retained E2F binding site is E2F I and/or E2F II. In certain embodiments, the retained E2F binding site is E2F II. In certain embodiments, the total deletion consists essentially of one or more of Pea3 II, Pea3 III, Pea3 IV, and/or Pea3 V. In certain embodiments, the virus has a deletion of a 50 base pair region located from −304 to −255 upstream of the E1a initiation site, e.g., corresponding to 195-244 of the Ad5 genome (SEQ ID NO:1), hereafter referred to as the TAV-255 deletion. In certain embodiments, the TAV-255 deletion results in an E1a promoter that comprises the sequence GGTGTTTTGG (SEQ ID NO:2).

In one embodiment, the recombinant adenovirus has the same or similar E1a modification as in the serotype 5 adenovirus (Ad5) called TAV-255 described in Publication No. WO 2010/101921 and U.S. Publication No. 2016-0017294, each of which is incorporated by reference herein in its entirety. It is believed that the mechanism by which the TAV-255 vector achieves transcriptional attenuation in normal cells is through targeted deletion of three transcriptional factor (TF) binding sites for the transcription factors Pea3 and E2F, proteins that regulate adenovirus expression of Ela, the earliest gene to be transcribed after virus entry into the host cell, through binding to specific DNA sequences. These three Pea3 and E2F deletions attenuate replication in growth-arrested, normal cells.

In one embodiment, the recombinant adenovirus comprises one or more Pea3 transcription binding site deletions without one or more E2F transcription binding site deletions in the E1A region. In other embodiment, the recombinant adenovirus comprises one or more E2F transcription binding site deletions without one or more Pea3 transcription binding site deletions in the E1A region.

In certain embodiments, the recombinant adenovirus comprises an E1a promoter lacking a functional TATA box, or lacking a functional CAAT box. In certain embodiments, the recombinant adenovirus comprises a deletion of the entire TATA box. In certain embodiments, the recombinant adenovirus comprises a deletion of the entire CAAT box.

The TATA box is recognized by Transcription Factor IIB (TFIIB) and the TATA binding protein (TBP), which are required for the recruitment of RNA pol II. The central role of the TATA box in transcription is supported by experimental observations of impaired or inactivated transcription following the mutation or removal of a TATA box, e.g., the removal of the TATA box in the promoter of the adenoviral E1a gene (Wu et al., Nature, 326(6112):512-515, 1987).

An additional sequence present in many promoters is a CAAT box. A CAAT box is typically located approximately 60-100 bases upstream of a gene's transcription start site and has the consensus sequence GG(T/C)CAATCT (SEQ ID NO:34). The CAAT box is recognized by core binding factors (also referred to as nuclear factor Y or NF-Y) and CCAAT/enhancer binding proteins (C/EBPs).

In certain embodiments, a recombinant adenovirus comprises an E1a promoter having a deletion of a functional TATA box, e.g., the deletion of an entire TATA box. As used herein, a “functional TATA box” refers to a TATA box that is capable of binding to a TATA box binding protein (TBP), e.g., a TATA box that has at least 100%, at least 90%, at least 80%, at least 70%, at least 60%, at least 50%, or at least 40%, of the TBP binding activity of a corresponding wild-type TATA box sequence. As used herein, a “non-functional TATA box” refers to a TATA box that, e.g., has less than 30%, less than 20%, less than 10%, or 0% of the TBP binding activity of a corresponding wild-type TATA box sequence. Assays for determining whether a TBP binds to a TATA box are known in the art. Exemplary binding assays include electrophoretic mobility shift assays, chromatin immunoprecipitation assays, and DNAse footprinting assays.

In certain embodiments, the recombinant adenovirus comprises a modified TATA box-based promoter may, e.g., comprise a deletion of the entire E1a promoter TATA box, e.g., comprise a deletion corresponding to nucleotides −27 to −24 of the Ad5 E1a promoter. For example, in certain embodiments, a recombinant adenovirus comprises a deletion of nucleotides corresponding to −27 to −24, −31 to −24, −44 to +54, or −146 to +54 of the adenovirus type 5 E1a promoter, which correspond, respectively, to nucleotides 472 to 475, 468 to 475, 455 to 552, and 353 to 552 of the Ad5 genome (SEQ ID NO:1). In certain embodiments, the virus comprises a polynucleotide deletion that results in a virus comprising the sequence CTAGGACTG (SEQ ID NO:17), AGTGCCCG (SEQ ID NO:16), or TATTCCCG (SEQ ID NO:15), which result from joining the two polynucleotide sequences that would otherwise flank the deleted polynucleotide sequence. In some embodiments, the virus may comprise a deletion of nucleotides corresponding to −29 to −26, −33 to −26, −44 to +52, or −148 to +52 upstream of the initiation site of Ela. In certain embodiments, the deletion comprises a deletion of nucleotides corresponding to 353-552 of the Ad5 genome (SEQ ID NO:1), and/or the E1a promoter comprises the sequence CTAGGACTG (SEQ ID NO:17).

In certain embodiments, a recombinant adenovirus may comprise an E1a promoter having a deletion of a functional CAAT box, e.g., the deletion of an entire CAAT box. As used herein, a “functional CAAT box” refers to a CAAT box that is capable of binding to a C/EBP or NF-Y protein, e.g., a CAAT box that has at least 100%, at least 90%, at least 80%, at least 70%, at least 60%, at least 50%, or at least 40%, of the a C/EBP or NF-Y binding activity of a corresponding wild-type CAAT box sequence. As used herein, a “non-functional CAAT box” refers to a CAAT box that, e.g., has less than 30%, less than 20%, less than 10%, or 0% of the a C/EBP or NF-Y binding activity of a corresponding wild-type CAAT box sequence. Assays for determining whether a C/EBP or NF-Y protein binds to a CAAT box are known in the art. Exemplary binding assays include electrophoretic mobility shift assays, chromatin immunoprecipitation assays, and DNAse footprinting assays. In certain embodiments, the recombinant adenovirus comprises a modified CAAT box-based promoter may, e.g., comprise a deletion of the entire E1a promoter CAAT box, e.g., comprise a deletion corresponding to nucleotides −76 to −68 of the adenovirus type 5 E1a promoter, which corresponds to nucleotides 423 to 431 of SEQ ID NO:1. In certain embodiments, the virus comprises a polynucleotide deletion that results in a virus comprising the sequence TTCCGTGGCG (SEQ ID NO:10), which results from joining the two polynucleotide sequences that would otherwise flank the deleted polynucleotide sequence.

B. Insertion Sites

In certain embodiments, the recombinant adenovirus comprises one or more nucleotide sequences comprising a transgene inserted in one of more of an E1b-19K insertion site, an E3 insertion site, an E4 insertion site, an IX-E2 insertion site, an L5-E4 insertion site, and combinations thereof.

In certain embodiments, the E1b-19K insertion site is located between the start site of E1b-19K and the start site of E1b-55K. The adenoviral E1b-19k gene functions primarily as an anti-apoptotic gene and is a homolog of the cellular anti-apoptotic gene, BCL-2. Since host cell death prior to maturation of the progeny viral particles would restrict viral replication, E1b-19k is expressed as part of the E1 cassette to prevent premature cell death thereby allowing the infection to proceed and yield mature virions. Accordingly, in certain embodiments, a recombinant virus is provided that includes an E1b-19K insertion site, e.g., the adenovirus has an exogenous nucleotide sequence inserted into an E1b-19K insertion site. In certain embodiments, the insertion site is located between the start site of E1b-19K and the stop codon of E1b-19K.

In certain embodiments, the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent to the start site of E1b-19K. In certain embodiments, the E1b-19K insertion site comprises a deletion of about 200 nucleotides, e.g., 202 nucleotides adjacent to the start site of E1b-19K. In certain embodiments, the E1b-19K insertion site comprises a deletion corresponding to nucleotides 1714-1917 of the Ad5 genome (SEQ ID NO:1), or, an exogenous nucleotide sequence encoding a transgene is inserted between nucleotides corresponding to 1714 and 1917 of the Ad5 genome (SEQ ID NO:1). In certain embodiments, an exogenous nucleotide sequence encoding a transgene is inserted between CTGACCTC (SEQ ID NO:4) and TCACCAGG (SEQ ID NO:5), e.g., the recombinant adenovirus comprises, in a 5′ to 3′ orientation, CTGACCTC (SEQ ID NO:4), an exogenous nucleotide sequence encoding a transgene, and TCACCAGG (SEQ ID NO:5). In some embodiments, the E1b-19K insertion site comprises a deletion of about 200 base pairs. The nucleotide sequence of the modified E1b-19k region is as follows, with residual bases from fused SalI and XhoI sites underlined:

(SEQ ID NO: 7)
ATCTTGGTTACATCTGACCTCGTCGAGTCACCAGGCGCTTTTCCAA

In certain embodiments, the E3 insertion site is located between the stop codon of pVIII and the start site of Fiber. In certain embodiments, the E3 insertion site is located between the stop codon of E3-10.5K and the stop codon of E3-14.7K and the start site of Fiber.

In certain embodiments, the E3 insertion site comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or about 3000 to about 3185 nucleotides. In certain embodiments, the E3 insertion site is located between the stop codon of E3-10.5K and the stop codon of E3-14.7K. In certain embodiments, the E3 insertion site comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop codon of E3-10.5K. In certain embodiments, the E3 insertion site comprises a deletion of about 1050 nucleotides adjacent the stop codon of E3-10.5K, e.g., the E3 insertion site comprises a deletion of 1063 nucleotides adjacent the stop codon of E3-10.5K. In certain embodiments, the E3 insertion site comprises a deletion corresponding to the Ad5 d1309 E3 deletion. In certain embodiments, the E3 insertion site comprises a deletion corresponding to nucleotides 29773-30836 of the Ad5 genome (SEQ ID NO:1), or a coronavirus coding region or expression cassette is inserted between nucleotides corresponding to 29773 and 30836 of the Ad5 genome (SEQ ID NO:1).

In certain embodiments, an E4 insertion site comprises any one of the ORF of the E4 gene. For example, a nucleotide sequence can be inserted in E4 ORF1, and/or E4 ORF2. In certain embodiments, portions of or the entire E4 region may be deleted.

In certain embodiments, the insertion site is the IX-E2 insertion site. In certain embodiments, the IX-E2 insertion site is located between the stop codon of adenovirus IX gene and the stop codon of adenovirus IVa2 gene. In certain embodiments, the nucleotide sequence is inserted between nucleotides corresponding to 4029 and 4093 of the Ad5 genome (SEQ ID NO:1). In certain embodiments, the nucleotide sequence is inserted between nucleotides corresponding to 4029 and 4050, nucleotides corresponding to 4051 and 4070, or nucleotides corresponding to 4071 and 4093 of the Ad5 genome (SEQ ID NO:1). In some embodiments, the IX-E2 insertion site has at least 60%, at least 70%, at least 80%, at least 90%, at least 95%, or at least 99% identity to nucleotides corresponding to 4029 and 4093 of the Ad5 genome (SEQ ID NO:1).

In certain embodiments, the insertion site is an L5-E4 insertion site. In certain embodiments, the L5-E4 insertion site is located between the stop codon of adenovirus fiber gene and the stop codon of ORF6 or ORF6/7 of the adenovirus E4 gene. In certain embodiments, the nucleotide sequence is inserted between nucleotides corresponding to 32785 to 32916 of the Ad5 genome (SEQ ID NO:1). In certain embodiments, the nucleotide sequence is inserted between nucleotides corresponding to 32785 and 32800, nucleotides corresponding to 32801 and 32820, nucleotides corresponding to 32821 and 32840, nucleotides corresponding to 32841 and 32860, nucleotides corresponding to 32861 and 32880, nucleotides corresponding to 32881 and 32900, or nucleotides corresponding to 32901 and 32916 of the Ad5 genome (SEQ ID NO:1). In some embodiments, the L5-E4 insertion site has at least 60%, at least 70%, at least 80%, at least 90%, at least 95%, or at least 99% identity to nucleotides corresponding to 32785 to 32916 of the Ad5 genome (SEQ ID NO:1).

In certain embodiments, the IX-E2 insertion site comprises a deletion of about 5, 10, 15, 20, 25, 30, 35, 40, 45, 50, 55, or 60 nucleotides. In certain embodiments, the L5-E4 insertion site comprises a deletion of about 5, 10, 15, 20, 25, 30, 35, 40, 45, 50, 55, 60, 65, 70, 75, 80, 85, 90, 95, 100, 105, 110, 115, 120, 125, or 130 nucleotides.

To accommodate insertion of a large coronavirus antigen or coronavirus antigen concatemers into the viral genome without exceeding the packaging capacity of an adenoviral capsid, compensatory deletions were made in the E3 and E4 regions. In the E3 region, the RID alpha, RID beta, and 14.7K genes positioned after adenoviral death protein were deleted, and the E3 gp19k gene was disrupted by mutating the fourth codon to a stop codon. The E4 region retained E4 ORF6/7. This virus was named PSV1.

In certain embodiments, the recombinant adenovirus comprises two or more nucleotide sequences, wherein the nucleotide sequences each comprises a transgene, wherein the nucleotide sequences are optionally separated by a linker. In certain embodiments, the recombinant adenovirus expresses two transgenes, when expressed, produce a single polypeptide chain, which may be cleaved post-translationally into two polypeptide chains. In certain embodiments, the linker is an internal ribosome entry site (IRES) element and/or a self-cleaving 2A peptide sequence. The IRES may, e.g., be selected from the group consisting of the encephalomyocarditis virus IRES, the foot-and-mouth disease virus IRES, and the poliovirus IRES.

In certain embodiments, the two or more nucleotide sequences are inserted in an E1b-19K insertion site, an E3 insertion site, an E4 insertion site, an IX-E2 insertion site, or an L5-E4 insertion site. In certain embodiments, the two or more nucleotide sequences are inserted in the same insertion site. In certain embodiments, the two or more nucleotide sequences are inserted in different insertion sites.

In certain embodiments, the nucleotide sequences encoding each coronavirus antigen are separated by an internal ribosome entry site (IRES), such as an encephalomyocarditis virus IRES, a foot-and-mouth disease virus IRES, or a poliovirus IRES. The nucleotide sequence of a representative IRES is:

(SEQ ID NO: 13)
TAACGTTACT GGCCGAAGCC GCTTGGAATA AGGCCGGTGT
GCGTTTGTCT ATATGTTATT TTCCACCATA TTGCCGTCTT
TTGGCAATGT GAGGGCCCGG AAACCTGGCC CTGTCTTCTT
GACGAGCATT CCTAGGGGTC TTTCCCCTCT CGCCAAAGGA
ATGCAAGGTC TGTTGAATGT CGTGAAGGAA GCAGTTCCTC
TGGAAGCTTC TTGAAGACAA ACAACGTCTG TAGCGACCCT
TTGCAGGCAG CGGAACCCCC CACCTGGCGA CAGGTGCCTC
TGCGGCCAAA AGCCACGTGT ATAAGATACA CCTGCAAAGG
CGGCACAACC CCAGTGCCAC GTTGTGAGTT GGATAGTTGT
GGAAAGAGTC AAATGGCTCT CCTCAAGCGT ATTCAACAAG
GGGCTGAAGG ATGCCCAGAA GGTACCCCAT TGTATGGGAT
CTGATCTGGG GCCTCGGTGC ACATGCTTTA CATGTGTTTA
GTCGAGGTTA AAAAACGTCT AGGCCCCCCG AACCACGGGG
ACGTGGTTTT CCTTTGAAAA ACACGATGAT AAT.

### II. Coronavirus Antigens

The disclosed recombinant vectors comprise an exogenous nucleotide sequence that encodes one or more coronavirus antigens or fragments thereof. As used herein, the term “antigen” refers a substance capable of being recognized and bound specifically by an antibody or by a T cell receptor. An antigen may additionally be capable of inducing a humoral immune response and/or a cellular immune response characterized by the elicitation of antigen-reactive B- and/or T-lymphocytes. Antigens can include, for example, polypeptides, proteins, glycoproteins, phosphoproteins, polysaccharides, gangliosides and lipids, portions thereof and combinations thereof. As used herein, the term antigen is understood to include a portion of an antigen that is bound specifically by an antibody or by a T cell receptor, also referred to as an “epitope.” Furthermore, it is understood that an epitope may be derived from a sequence of substituents that are consecutive or non-consecutive, for example, in a protein, it is understood that an epitope may be defined by the primary sequence of amino acids in the protein and/or by the tertiary structure of the protein.

In preferred embodiments, coronavirus antigens of the present disclosure are polypeptide antigens or fragments thereof that are at least eight amino acid residues in length, preferably from 8 to 1800 amino acids in length, or from 15 to 1500 amino acids in length, or from 20 to 1200 amino acids in length. In some embodiments, the polypeptide is at least about (lower limit) 8, 9, 10, 11, 12, 13, 14, 15, 20, 25, 30, 35, 40, 45, 50, 60, 70, 80 90 or 100 amino acids in length. In some embodiments, the polypeptide is at most (upper limit) about 1800, 1700, 1600, 1500, 1400, 1300, 1200, 1100, 1000, 900, 800, 700, 600, 500, 400, 300 or 200 amino acids in length.

Coronavirus antigens expressed by infection of host cells with recombinant adenoviruses of the present disclosure include coronavirus structural proteins. For instance the recombinant adenovirus may comprise a nucleotide sequence encoding a coronavirus nucleocapsid protein, spike protein, membrane protein, envelope protein, fragments thereof, or combinations thereof. In some embodiments, the coronavirus antigen comprises a nucleocapsid (N) protein. In some embodiments, the coronavirus antigen comprises a spike (S) protein or the receptor-binding domain (RBD) of the spike protein. In some embodiments, the coronavirus antigen comprises one or both of a membrane (M) protein and an envelope (E) protein. In some embodiments, the coronavirus antigen comprises or further comprises a non-structural protein.

In certain embodiments, the nucleotide sequences encoding each coronavirus antigen are separated by a nucleotide sequence encoding a protein linker, preferably a protein linker comprising a cleavage site. In some embodiments, the amino acid sequence of the protein linker comprises AAY (SEQ ID NO:6).

In some aspects, the linker comprises a cleavage site, e.g., a proteolytic or a non-proteolytic cleavage site, or a ribosome skipping sequence, e.g., a T2A sequence. In certain embodiments, the multiple nucleotide sequences each encoding a coronavirus antigen are separated by a proteolytic cleavage site. In certain embodiments, the proteolytic cleavage site is cleaved by a protease present in a specific tissue, organelle or intracellular compartment. In certain embodiments, the linker comprises a proteolytic cleavage site and two cysteine residues that result in a disulfide linkage following proteolytic cleavage. In certain embodiments, the proteolytic cleavage site is cleaved by a protease selected from a matrix metalloproteinase (MMP), furin, PC1, PC2, PC3, cathepsin B, proteinase 3, and caspase 3.

In certain embodiments, the cleavage site is a proteolytic cleavage site that is cleaved by a protease that is present in the endoplasmic reticulum or golgi of a eukaryotic cell. In certain embodiments, the proteolytic cleavage site is a furin cleavage site. Furin is a protease that is ubiquitously expressed and is localized to the Golgi, where it recognizes the consensus sequence RX₁X₂R (SEQ ID NO:18), wherein X₁ is any amino acid, and X₂ is lysine or arginine, and cleaves after the final arginine residue. Furin plays a biological role in cleaving propeptides of proteins that are trafficked through the Golgi. Accordingly, in certain embodiments the proteolytic cleavage site is a furin cleavage site comprising the sequence RX₁X₂R (SEQ ID NO: 18), wherein X₁ is any amino acid, and X₂ is lysine or arginine. In some embodiments, the furin cleavage site comprising the sequence RAKR (SEQ ID NO:19).

In certain embodiments, wherein a recombinant vector e.g., a recombinant oncolytic vector, comprises multiple nucleotide sequences, each of which encodes a coronavirus antigen, for example, wherein the recombinant vector comprises a nucleotide sequence encoding a single polypeptide chain comprising multiple coronavirus antigens, each separated by a protein linker, the recombinant vector may further comprise a nucleotide sequence encoding ubiquitin to enhance proteolysis of the single polypeptide chain (see, Velders et al. (2001) J. Immunol. 166: 5366-5373, the contents of which are incorporated herein by reference in its entirety).

In some aspects, the exogenous nucleotide sequence comprising one or more nucleotide sequences, each encoding a coronavirus antigen, is inserted into one insertion site selected from the group consisting of E1b-19K insertion site, E3 insertion site, E4 insertion site, IX-E2 insertion site, and L5-E4 insertion site, wherein each of the nucleotide sequences is separated from each other by at least one linker. In some aspects, multiple nucleotide sequences, each encoding a coronavirus antigen, are inserted in such a manner that they are distributed among 2 or more insertion sites selected from the following insertion sites—E1b-19K insertion site, E3 insertion site, E4 insertion site, IX-E2 insertion site, and L5-E4 insertion site.

In some aspects, the coronavirus is an alphacoronavirus, a betacoronavirus, a gammacoronavirus, or a deltacoronavirus. In some aspects, the coronavirus is gammacoronavirus. In some embodiments, the coronavirus is a severe acute respiratory syndrome coronavirus 2 (SARS-CoV-2). In other embodiments, the coronavirus is a severe acute respiratory syndrome coronavirus (SARS-CoV). In further embodiments, the coronavirus is a middle east respiratory syndrome-related coronavirus (MERS-CoV). In some embodiments, the gammacoronavirus is SARS-CoV-2, SARS-CoV-1, or MERS-CoV.

The nucleotide sequence of a representative SARS-CoV-2 isolate (Wuhan-Hu-1) is set forth as GenBank No. MN908947.3 (Wu et al., Nature, 579:265-269, 2020).

The amino acid sequence of a representative SARS-CoV-2 N protein is:

(SEQ ID NO: 21)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADL
DDFSKQLQQSMSSADSTQA.

The amino acid sequence of a representative SARS-CoV-2 S protein is:

(SEQ ID NO: 22)
MEVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNEKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT.

In some preferred embodiments, the SARS-CoV-2 antigen comprises the receptor-binding domain (RBD) of the S protein:

(SEQ ID NO: 23)
NSNNLDSKVGGNYNYLYRLFRKSNLKPFERDISTEIYQAGSTPCNGVEGF
NCYFPLQSYGFQPTNGVGYQPYR.

The amino acid sequence of a representative SARS-CoV-2 M protein is:

(SEQ ID NO: 24)
MADSNGTITVEELKKLLEQWNLVIGFLFLTWICLLQFAYANRNRFLYIIK
LIFLWLLWPVTLACFVLAAVYRINWITGGIAIAMACLVGLMWLSYFIASF
RLFARTRSMWSFNPETNILLNVPLHGTILTRPLLESELVIGAVILRGHLR
IAGHHLGRCDIKDLPKEITVATSRTLSYYKLGASQRVAGDSGFAAYSRYR
IGNYKLNTDH SSSSDNIALLVQ.

The amino acid sequence of a representative SARS-CoV-2 E protein is:

(SEQ ID NO: 25)
MYSFVSEETGTLIVNSVLLFLAFVVFLLVTLAILTALRLCAYCCNIVNVS
LVKPSFYVYSRVKNLNSSRVPDLLV.

The amino acid sequence of a representative SARS-CoV-1 N protein is:

(SEQ ID NO: 26)
MSDNGPQSNQRSAPRITFGGPTDSTDNNQNGGRNGARPKQRRPQGLPNNT
ASWFTALTQHGKEELRFPRGQGVPINTNSGPDDQIGYYRRATRRVRGGDG
KMKELSPRWYFYYLGTGPEASLPYGANKEGIVWVATEGALNTPKDHIGTR
NPNNNAATVLQLPQGTTLPKGFYAEGSRGGSQASSRSSSRSRGNSRNSTP
GSSRGNSPARMASGGGETALALLLLDRLNQLESKVSGKGQQQQGQTVTKK
SAAEASKKPRQKRTATKQYNVTQAFGRRGPEQTQGNFGDQDLIRQGTDYK
HWPQIAQFAPSASAFFGMSRIGMEVTPSGTWLTYHGAIKLDDKDPQFKDN
VILLNKHIDAYKTFPPTEPKKDKKKKTDEAQPLPQRQKKQPTVTLLPAAD
MDDFSRQLQNSMSGASADSTQA.

The amino acid sequence of a representative SARS-CoV-1 S protein is:

(SEQ ID NO: 27)
MFIFLLFLTLTSGSDLDRCTTFDDVQAPNYTQHTSSMRGVYYPDEIFRSD
TLYLTQDLFLPFYSNVTGFHTINHTFGNPVIPFKDGIYFAATEKSNVVRG
WVFGSTMNNKSQSVIIINNSTNVVIRACNFELCDNPFFAVSKPMGTQTHT
MIFDNAFNCTFEYISDAFSLDVSEKSGNFKHLREFVFKNKDGFLYVYKGY
QPIDVVRDLPSGFNTLKPIFKLPLGINITNFRAILTAFSPAQDIWGTSAA
AYFVGYLKPTTFMLKYDENGTITDAVDCSQNPLAELKCSVKSFEIDKGIY
QTSNFRVVPSGDVVRFPNITNLCPFGEVFNATKFPSVYAWERKKISNCVA
DYSVLYNSTFFSTFKCYGVSATKLNDLCFSNVYADSFVVKGDDVRQIAPG
QTGVIADYNYKLPDDFMGCVLAWNTRNIDATSTGNYNYKYRYLRHGKLRP
FERDISNVPFSPDGKPCTPPALNCYWPLNDYGFYTTTGIGYQPYRVVVLS
FELLNAPATVCGPKLSTDLIKNQCVNFNFNGLTGTGVLTPSSKRFQPFQQ
FGRDVSDFTDSVRDPKTSEILDISPCSFGGVSVITPGTNASSEVAVLYQD
VNCTDVSTAIHADQLTPAWRIYSTGNNVFQTQAGCLIGAEHVDTSYECDI
PIGAGICASYHTVSLLRSTSQKSIVAYTMSLGADSSIAYSNNTIAIPTNF
SISITTEVMPVSMAKTSVDCNMYICGDSTECANLLLQYGSFCTQLNRALS
GIAAEQDRNTREVFAQVKQMYKTPTLKYFGGFNFSQILPDPLKPTKRSFI
EDLLFNKVTLADAGFMKQYGECLGDINARDLICAQKFNGLTVLPPLLTDD
MIAAYTAALVSGTATAGWTFGAGAALQIPFAMQMAYRFNGIGVTQNVLYE
NQKQIANQFNKAISQIQESLTTTSTALGKLQDVVNQNAQALNTLVKQLSS
NFGAISSVLNDILSRLDKVEAEVQIDRLITGRLQSLQTYVTQQLIRAAEI
RASANLAATKMSECVLGQSKRVDFCGKGYHLMSFPQAAPHGVVFLHVTYV
PSQERNFTTAPAICHEGKAYFPREGVFVFNGTSWFITQRNFFSPQIITTD
NTFVSGNCDVVIGIINNTVYDPLQPELDSFKEELDKYFKNHTSPDVDLGD
ISGINASVVNIQKEIDRLNEVAKNLNESLIDLQELGKYEQYIKWPWYVWL
GFIAGLIAIVMVTILLCCMTSCCSCLKGACSCGSCCKFDEDDSEPVLKGV
KLHYT.

The amino acid sequence of a representative SARS-CoV-1 M protein is:

(SEQ ID NO: 28)
MADNGTITVEELKQLLEQWNLVIGFLFLAWIMLLQFAYSNRNRFLYIIKL
VFLWLLWPVTLACFVLAAVYRINWVTGGIAIAMACIVGLMWLSYFVASFR
LFARTRSMWSFNPETNILLNVPLRGTIVTRPLMESELVIGAVIIRGHLRM
AGHSLGRCDIKDLPKEITVATSRTLSYYKLGASQRVGTDSGFAAYNRYRI
GNYKLNTDHAGSNDNIALLVQ.

The amino acid sequence of a representative SARS-CoV-1 E protein is:

(SEQ ID NO: 29)
MYSFVSEETGTLIVNSVLLFLAFVVFLLVTLAILTALRLCAYCCNIVNVS
LVKPTVYVYSRVKNLNSSEGVPDLLV.

The amino acid sequence of a representative MERS-CoV N protein is:

(SEQ ID NO: 30)
MASPAAPRAVSFADNNDITNTNLSRGRGRNPKPRAAPNNTVSWYTGLTQH
GKVPLTFPPGQGVPLNANSTPAQNAGYWRRQDRKINTGNGIKQLAPRWYF
YYTGTGPEAALPFRAVKDGIVWVHEDGATDAPSTFGTRNPNNDSAIVTQF
APGTKLPKNFHIEGTGGNSQSSSRASSVSRNSSRSSSQGSRSGNSTRGTS
PGPSGIGAVGGDLLYLDLLNRLQALESGKVKQSQPKVITKKDAAAAKNKM
RHKRTSTKSFNMVQAFGLRGPGDLQGNFGDLQLNKLGTEDPRWPQIAELA
PTASAFMGMSQFKLTHQNNDDHGNPVYFLRYSGAIKLDPKNPNYNKWLEL
LEQNIDAYKTFPKKEKKQKAPKEESTDQMSEPPKEHRVQGTQRTRTRPSV
QPGPMIDVNTD.

The amino acid sequence of a representative MERS-CoV S protein is:

(SEQ ID NO: 31)
MIHSVFLLMFLLTPTESYVDVGPDSVKSACIEVDIQQTFFDKTWPRPIDV
SKADGIIYPQGRTYSNITITYQGLFPYQGDHGDMYVYSAGHATGTTPQKL
FVANYSQDVKQFANGFVVRIGAAANSTGTVIISPSTSATIRKIYPAFMLG
SSVGNFSDGKMGRFFNHTLVLLPDGCGTLLRAFYCILEPRSGNHCPAGNS
YTSFATYHTPATDCSDGNYNRNASLNSFKEYFNLRNCTFMYTYNITEDEI
LEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCS
YESFDVESGVYSVSSFEAKPSGSVVEQAEGVECDFSPLLSGTPPQVYNFK
RLVFTNCNYNLTKLLSLFSVNDFTCSQISPAAIASNCYSSLILDYFSYPL
SMKSDLSVSSAGPISQFNYKQSFSNPTCLILATVPHNLTTITKPLKYSYI
NKCSRFLSDDRTEVPQLVNANQYSPCVSIVPSTVWEDGDYYRKQLSPLEG
GGWLVASGSTVAMTEQLQMGFGITVQYGTDTNSVCPKLEFANDTKIASQL
GNCVEYSLYGVSGRGVFQNCTAVGVRQQRFVYDAYQNLVGYYSDDGNYYC
LRACVSVPVSVIYDKETKTHATLFGSVACEHISSTMSQYSRSTRSMLKRR
DSTYGPLQTPVGCVLGLVNSSLFVEDCKLPLGQSLCALPDTPSTLTPRSV
RSVPGEMRLASIAFNHPIQVDQLNSSYFKLSIPTNFSFGVTQEYIQTTIQ
KVTVDCKQYVCNGFQKCEQLLREYGQFCSKINQALHGANLRQDDSVRNLF
ASVKSSQSSPIIPGFGGDFNLTLLEPVSISTGSRSARSAIEDLLFDKVTI
ADPGYMQGYDDCMQQGPASARDLICAQYVAGYKVLPPLMDVNMEAAYTSS
LLGSIAGVGWTAGLSSFAAIPFAQSIFYRLNGVGITQQVLSENQKLIANK
FNQALGAMQTGFTTTNEAFHKVQDAVNNNAQALSKLASELSNTFGAISAS
IGDIIQRLDVLEQDAQIDRLINGRLTTLNAFVAQQLVRSESAALSAQLAK
DKVNECVKAQSKRSGFCGQGTHIVSFVVNAPNGLYFMHVGYYPSNHIEVV
SAYGLCDAANPTNCIAPVNGYFIKTNNTRIVDEWSYTGSSFYAPEPITSL
NTKYVAPQVTYQNISTNLPPPLLGNSTGIDFQDELDEFFKNVSTSIPNFG
SLTQINTTLLDLTYEMLSLQQVVKALNESYIDLKELGNYTYYNKWPWYIW
LGFIAGLVALALCVFFILCCTGCGTNCMGKLKCNRCCDRYEEYDLEPHKV
HVH.

The amino acid sequence of a representative MERS-CoV M protein is:

(SEQ ID NO: 32)
MSNMTQLTEAQIIAIIKDWNFAWSLIFLLITIVLQYGYPSRSMTVYVFKM
FVLWLLWPSSMALSIFSAVYPIDLASQIISGIVAAVSAMMWISYFVQSIR
LFMRTGSWWSFNPETNCLLNVPFGGTTVVRPLVEDSTSVTAVVTNGHLKM
AGMHFGACDYDRLPNEVTVAKPNVLIALKMVKRQSYGTNSGVAIYHRYKA
GNYRSPPITADIELALLRA.

The amino acid sequence of a representative MERS-CoV E protein is:

(SEQ ID NO: 33)
MLPFVQERIGLFIVNFFIFTVVCAITLLVCMAFLTATRLCVQCMTGENTL
LVQPALYLYNTGRSVYVKFQDSKPPLPPDEWV.

In some embodiments, the coronavirus antigen is a variant of a representative coronavirus structural protein disclosed herein. In some embodiments, the amino acid sequence of the variant antigen is at least 75%, 85%, 90%, 95%, 96%, 97%, 98% or 99% identical to the amino acid sequence of the representative antigen. Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) Proc. Natl. Acad. Sci. USA 87:2264-2268; Altschul, (1993) J. Mol. Evol. 36, 290-300; Altschul et al., (1997) Nucleic Acids Res. 25:3389-3402, incorporated by reference) are tailored for sequence similarity searching. For a discussion of basic issues in searching sequence databases see Altschul et al., (1994) Nature Genetics 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) Proc. Natl. Acad. Sci. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; −W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

### III. Methods for Virus Production

Nucleic acids encoding coronavirus antigens can be incorporated into plasmids and introduced into host cells through conventional transfection or transformation techniques. Specific production and purification conditions will vary depending upon the virus and the production system employed. For adenovirus, the traditional method for the generation of viral particles is co-transfection followed by subsequent in vivo recombination of a shuttle plasmid (usually containing a small subset of the adenoviral genome and optionally containing a potential transgene an expression cassette) and an adenoviral helper plasmid (containing most of the entire adenoviral genome).

Recombinant adenoviruses and method of making and using them are described in U.S. application Ser. No. 15/991,745, U.S. application Ser. No. 16/058,886, PCT/US2018/034888, and PCT/US2018/030929, each of which is incorporated by reference in its entirety.

### IV. Pharmaceutical Compositions

For prophylactic or therapeutic use, a recombinant adenovirus disclosed herein is preferably combined with a pharmaceutically acceptable excipient. Likewise for prophylactic or therapeutic use, a coronavirus immune globulin preparation disclosed herein is preferably combined with a pharmaceutically acceptable excipient. As used herein, prophylactic and therapeutic uses include preclinical and clinical uses. Pharmaceutically acceptable excipients of the present disclosure include for instance, solvents, bulking agents, buffering agents, tonicity adjusting agents, and preservatives (see, e.g., Pramanick et al., Pharma Times, 45:65-77, 2013). In some embodiments the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent). As used herein, “pharmaceutically acceptable” means the excipient is suitable for use in contact with the tissues of humans and other mammalian subjects without excessive toxicity, irritation, allergic response, or other problem or complication, commensurate with a reasonable benefit/risk ratio. The excipient should be “acceptable” in the sense of being compatible with other ingredients of the formulation and not deleterious to the recipient.

Pharmaceutical compositions can be provided in a dosage unit form and can be prepared by any suitable method. A pharmaceutical composition should be formulated to be compatible with its intended route of administration. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method (e.g., filtration through sterile filtration membranes). Where the composition is lyophilized, filter sterilization can be conducted prior to or following lyophilization and reconstitution.

The term “effective amount” as used herein refers to the amount of an active component (e.g., recombinant human adenovirus or coronavirus immune globulin preparation) sufficient to effect beneficial or desired results. An effective amount can be administered in one or more administrations, applications or dosages and is not intended to be limited to a particular formulation or administration route.

In certain embodiments, an effective amount of active agent is in the range of 0.1 mg/kg to 100 mg/kg, preferably 0.5 mg/kg to 20 mg/kg, or preferably 1 mg/kg to 10 mg/kg. The amount administered will depend on variables such as the type and extent of disease or indication to be treated or prevented, the overall health of the patient, the in vivo potency of the active agent, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized for instance in a conventional Phase I dose escalation study. Dosing frequency can vary, depending on factors such as route of administration, dosage amount, and serum half-life of the active agent. In certain embodiments, an effective amount of a recombinant adenovirus is in the range of 10² to 10¹⁵ plaque forming units (pfus) (e.g., 10² to 10¹⁰, 10² to 10⁵, 10⁵ to 10¹⁵, 10⁵ to 10¹⁰, or 10¹⁰ to 10¹⁵ pfus).

### V. Methods of Use

The present disclosure relates to methods for stimulating an immune response against a coronavirus in a human subject, methods for treating long COVID-19 in a human subject, and methods for treating cancer in a human subject.

In particular, the present disclosure relates to methods for stimulating an immune response against a coronavirus in a human subject, comprising administering an effective amount of the recombinant human adenovirus as described herein to a human subject so as to stimulate an immune response against a structural protein of the coronavirus in the human subject. In some embodiments, the structural protein comprises a nucleocapsid protein, a spike protein, a membrane protein, an envelope protein, a fragment thereof, or a combination thereof. In some embodiments, the structural protein comprises a nucleocapsid protein. In some embodiments, the structural protein comprises a spike protein. In some embodiments, the structural protein comprises a spike protein, a membrane protein, and an envelope protein. Stimulating an immune response, means increasing the immune response, which can arise from eliciting a de novo immune response (e.g., as a consequence of an initial vaccination regimen) or enhancing an existing immune response (e.g., as a consequence of a booster vaccination regimen). In some embodiments, the immune response is a coronavirus structural protein-reactive immune response and stimulating the immune response includes but is not limited to one or more of the group consisting of: stimulating cytokine production; stimulating B lymphocyte proliferation; stimulating antibody production; and stimulating cytotoxic T lymphocyte activity.

Additionally, the present disclosure relates to methods for treating cancer in a human subject comprising administering an effective amount of the recombinant human adenovirus as described herein to a human subject with a solid tumor. “Treating” cancer means to bring about a beneficial clinical result such as causing remission or otherwise prolonging survival as compared to expected survival in the absence of treatment. In some embodiments, “treating” cancer comprises shrinking the size of a tumor or otherwise reducing viable cancer cell numbers. In other embodiments, “treating” cancer comprises delaying growth of a tumor. In some embodiments, the recombinant adenovirus is administered by intra-tumoral or peri-tumoral delivery.

### Enumerated Embodiments

1. A recombinant human adenovirus for stimulating an immune response against a coronavirus in a human subject, wherein

the adenovirus comprises a nucleotide sequence encoding a structural protein of a coronavirus located at an insertion site in the adenovirus genome,

the coronavirus structural protein comprises a nucleocapsid protein, a spike protein, a membrane protein, an envelope protein, a fragment thereof, or a combination thereof, and

the adenovirus genome comprises a modified E1a transcription regulatory sequence.

2. The recombinant human adenovirus of embodiment 1, wherein the recombinant adenovirus is a type 5 adenovirus (Ad5).

3. The recombinant human adenovirus of embodiment 1 or embodiment 2, wherein the coronavirus is an alphacoronavirus, a betacoronavirus, a gammacoronavirus, or a deltacoronavirus, optionally wherein the coronavirus is gammacoronavirus selected from the group consisting of a severe acute respiratory syndrome coronavirus 2 (SARS-CoV-2), a severe acute respiratory syndrome coronavirus 1 (SARS-CoV-1), and a middle east respiratory syndrome-related coronavirus (MERS-CoV), optionally wherein the coronavirus is a SARS-CoV-2.

4. The recombinant human adenovirus of any one of embodiments 1-3, wherein the insertion site is selected from the group consisting of an E1b-19K insertion site, an E3 insertion site, an E4 insertion site, an IX-E2 insertion site, an L5-E4 insertion site, and combinations thereof.

5. The recombinant human adenovirus of any one of embodiments 1-4, wherein the modified E1a transcription regulatory sequence is a modified E1a promoter.

6. The recombinant human adenovirus of embodiment 5, wherein the modified E1a promoter comprises a deletion of nucleotides corresponding to 195-244 of the Ad5 genome (SEQ ID NO:1).

7. The recombinant human adenovirus of embodiment 6, wherein the modified E1a promoter comprises the sequence GGTGTTTTGG (SEQ ID NO:2).

8. The recombinant human adenovirus of any one of embodiments 1-7, wherein the coronavirus structural protein comprises a coronavirus nucleocapsid protein or a fragment thereof.

9. The recombinant human adenovirus of embodiment 8, wherein the nucleotide sequence encoding the nucleocapsid protein or a fragment thereof is located at the E1b-19K insertion site between the start site of E1b-19K and the stop codon of E1b-19K in place of about 200 nucleotides of E1b-19K.

10. The recombinant human adenovirus of embodiment 9, wherein the E1b-19K insertion site is between nucleotides corresponding to 1714 and 1916 of the Ad5 genome (SEQ ID NO:1).

11. The recombinant human adenovirus of embodiment 9, wherein the nucleotide sequence encoding the nucleocapsid protein of a fragment thereof is inserted between CTGACCTC (SEQ ID NO:4) and TCACCAGG (SEQ ID NO:5).

12. The recombinant human adenovirus of any one of embodiments 1-11, wherein the adenovirus genome comprises a deletion of at least a portion of E3.

13. The recombinant human adenovirus of any one of embodiments 1-12, wherein the coronavirus structural protein comprises a coronavirus spike protein or a fragment thereof.

14. The recombinant human adenovirus of embodiment 13, wherein the nucleotide sequence encoding the spike protein or fragment thereof is located at the L5-E4 insertion site between the stop codon of adenovirus fiber gene and the stop codon of adenovirus E4-ORF6/7 gene.

15. The recombinant human adenovirus of embodiment 14, wherein the L5-E4 insertion site is between nucleotides corresponding to 32787 and 32914 of the Ad5 genome (SEQ ID NO:1).

16. The recombinant human adenovirus of embodiment 14, wherein the nucleotide sequence encoding the spike protein or a fragment thereof is contained within an expression cassette comprising residues 24-543 of the sequence of SEQ ID NO:14.

17. The recombinant human adenovirus of any one of embodiments 1-16, wherein the coronavirus structural protein comprises a coronavirus membrane protein or a fragment thereof.

18. The recombinant human adenovirus of embodiment 17, wherein the nucleotide sequence encoding the membrane protein or fragment thereof is located at the IX-E2 insertion site between the stop codon of adenovirus IX gene and the stop codon of adenovirus IVa2 gene.

19. The recombinant human adenovirus of any one of embodiments 1-18, wherein the coronavirus structural protein comprises a coronavirus envelope protein or a fragment thereof.

20. The recombinant human adenovirus of embodiment 19, wherein the nucleotide sequence encoding the envelope protein or fragment thereof is located at the IX-E2 insertion site between the stop codon of adenovirus IX gene and the stop codon of adenovirus IVa2 gene.

21. The recombinant human adenovirus of embodiment 18 or embodiment 20, wherein:

(i′) the IX-E2 insertion site is between nucleotides corresponding to 4029 and 4093 of the Ad5 genome (SEQ ID NO:1); and/or

(ii′) the nucleotide sequence encoding the membrane protein or fragment thereof is contained within an expression cassette comprising residues 33-478 of the sequence of SEQ ID NO:20; or

(iii′) the nucleotide sequence encoding the envelope protein or a fragment thereof is contained within an expression cassette comprising residues 479-727 of the sequence of SEQ ID NO:20; or

(iv′) the nucleotide sequence encoding the envelope protein or a fragment thereof and the nucleotide sequence encoding the membrane protein or a fragment thereof are contained within an expression cassette comprising residues 33-727 of the sequence of SEQ ID NO:20.

22. The recombinant human adenovirus of any one of embodiments 1-21, wherein the adenovirus genome comprises a deletion of at least a portion of E4.

23. A kit comprising:

i) the recombinant human adenovirus of any one of embodiments 1-22, and

ii) instructions for administration of the adenovirus to stimulate an immune response against the coronavirus structural antigen in the human subject.

24. The kit of embodiment 23, further comprising a syringe and needle for injection of the recombinant adenovirus, optionally by intramuscular, subcutaneous, intradermal, intratumoral or intravenous injection.

25. A method for stimulating an immune response against a coronavirus in a human subject, comprising administering an effective amount of the recombinant human adenovirus of any one of embodiments 1-22 to a human subject so as to stimulate an immune response against the coronavirus structural antigen in the human subject.

26. The method of embodiment 25, wherein the recombinant human adenovirus is administered by intramuscular or subcutaneous injection.

27. The method of embodiment 25 or embodiment 26, wherein stimulating an immune response comprises eliciting a coronavirus neutralizing antibody response.

28. The method of any one of embodiments 25-27, wherein stimulating an immune response comprises eliciting one or both of a coronavirus-reactive CD4+ helper T cell response and a coronavirus-reactive CD8+ cytotoxic T lymphocyte response.

29. A method for preparing a coronavirus immune globulin preparation, the method comprising:

(a) immunizing a plurality of healthy adult human subjects between the ages of 18-60 with an effective amount of the recombinant human adenovirus of any one of embodiments 1-22 to elicit coronavirus neutralizing antibodies;

(b) harvesting plasma from the immunized subjects, optionally wherein the plasma is harvested by plasmapheresis;

(c) pooling the plasma to obtain a pooled plasma preparation comprising the coronavirus neutralizing antibodies; and

(d) fractionating the pooled plasma preparation to obtain coronavirus immune globulin preparation.

30. The method of embodiment 29, further comprising preparing an IgG coronavirus immune globulin preparation by subjecting the immune globulin preparation to affinity chromatography.

31. The method of embodiment 30, further comprising preparing an IgG isotype-enriched coronavirus immune globulin preparation by enriching the IgG coronavirus immune globulin preparation in one or both of IgG2 and IgG4 by one or both of

(i) positive selection of one or both of IgG2 and IgG4; and

(ii) negative selection of one or both of IgG1 and IgG3.

32. The method of embodiment 30, further comprising preparing an Fc-depleted coronavirus immune globulin preparation by:

(i) subjecting the IgG coronavirus immune globulin preparation to proteolysis with either pepsin to produce F(ab′)2 and Fc fragments or with papain to produce Fab and Fc; and

(ii) removal of the Fc or fragments thereof.

33. The method of any one of embodiments 29-32, further comprising (e) processing the coronavirus immune globulin preparation to obtain a formulation suitable for injection.

34. A coronavirus immune globulin preparation prepared according to the method of any one of embodiments 29-33.

35. A method of treating coronavirus disease in a human subject, comprising administering to the subject a therapeutically effective amount of the coronavirus immune globulin preparation of embodiment 34.

36. A method of providing immunotherapy to a human subject, comprising administering to the subject a therapeutically effective amount of the coronavirus immune globulin preparation of embodiment 34.

37. The method of embodiment 35 or embodiment 36, wherein the coronavirus immune globulin preparation is administered to the subject by intravenous infusion.

38. A method for treating long COVID-19 in a human subject, comprising administering an effective amount of the recombinant human adenovirus of any one of embodiments 1-22 to a human subject with long COVID-19.

39. The method of embodiment 38, wherein treating long COVID-19 comprises reducing duration or severity of COVID-19 symptoms experienced by the subject at baseline.

40. The method of embodiment 38, wherein treating long COVID-19 comprises alleviating one or more of fatigue, cognitive dysfunction, and sleep disturbance experienced by the subject at baseline.

41. The method of embodiment 38, wherein treating long COVID-19 comprises reducing duration of or likelihood of hospitalization for COVID-19.

42. A method for treating cancer in a human subject, comprising administering an effective amount of the recombinant human adenovirus of any one of embodiments 1-22 to a human subject with a solid tumor.

43. The method of embodiment 42, wherein treating cancer comprises improving overall objective response as determined according to response evaluation criteria in solid tumors (RECIST).

44. The method of embodiment 42, wherein treating cancer comprises reducing size of the solid tumor as measured by magnetic resonance imaging.

45. The method of embodiment 42, wherein treating cancer comprises retarding growth of the solid tumor as measured by magnetic resonance imaging.

46. Use of the coronavirus immune globulin preparation prepared according to the method of embodiment 29 for treating coronavirus disease in a human subject or for providing immunotherapy to a human subject, the method comprising administering to the subject an effective amount of the coronavirus immune globulin preparation.

47. Use of the recombinant human adenovirus of any one of embodiments 1-22 for stimulating an immune response against a coronavirus in a human subject, the method comprising administering to the subject an effective amount of the recombinant human adenovirus.

48. Use of the recombinant human adenovirus of any one of embodiments 1-22 for treating long COVID-19 in a human subject, the method comprising administering an effective amount of the recombinant human adenovirus.

49. Use of the recombinant human adenovirus of any one of embodiments 1-22 for treating cancer in a human subject, the comprising administering an effective amount of the recombinant human adenovirus.

50. A pharmaceutical composition comprising a pharmaceutically acceptable excipient and the recombinant human adenovirus of any one of embodiments 1-22.

### EXAMPLES

The following examples are merely illustrative of certain embodiments and are not intended to limit the scope of the disclosure.

### Example 1. Mono- and Multi-Valent SARS-CoV-2 Adenoviral Vectors

This example describes the production of recombinant human adenovirus type 5 (Ad5) vectors engineered to express one or more SARS-CoV-2 antigens. The human Ad5 vectors are live, transcriptionally-attenuated viruses. The nucleotide sequence of a representative human Ad5 genome is set forth below as SEQ ID NO:1.

To generate nVAX-19, sVAX-19, and Geist-20, plasmids each carrying partially overlapping segments of approximately one quarter of the human adenovirus type 5 genome flanked by Pad restriction sites were modified using standard molecular biology techniques. The base plasmids carried the TAV-255 deletion in the viral E1A promoter.

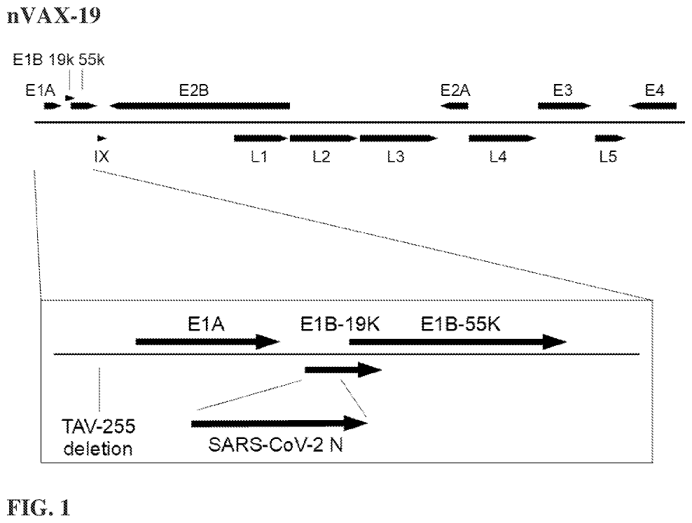

For nVAX-19, the plasmids were based on strain d1309, which carries a disruption in the adenoviral E3 region. SalI and XhoI restriction sites were introduced at the beginning of the E1B-19K gene and approximately 200 nucleotides downstream (within the E1B-19K coding region and before the start of the E1B-55K coding region), respectively. The plasmid was digested with SalI and XhoI and cDNA encoding SARS-CoV-2 nucleocapsid (N) was cloned into the site. A schematic of the nVAX-19 genome is shown in FIG. 1.

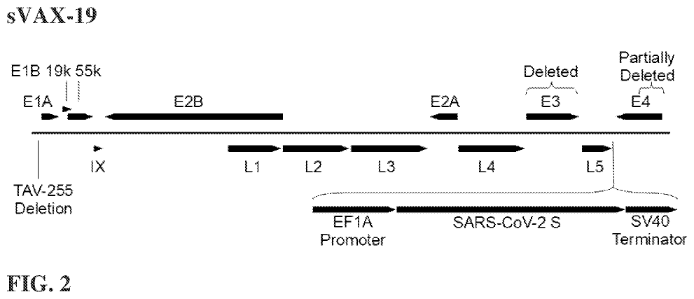

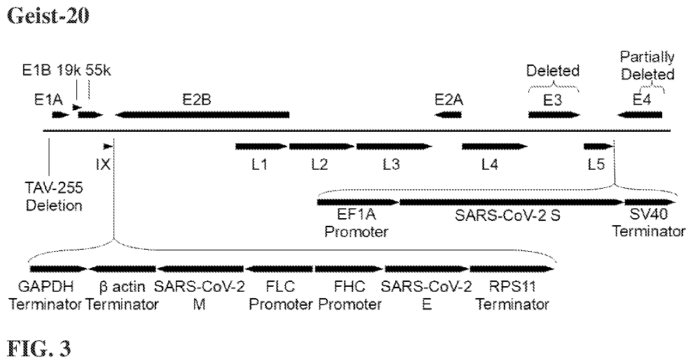

For sVAX-19 and Geist-20, due to the large size of the SARS-CoV-2 S (spike) gene, the plasmids carried deletions of the adenoviral E3 region and part of the adenoviral E4 region leaving only E4 ORF6/7 intact. For sVAX-19, an expression cassette with the EF1A promoter, cDNA encoding the SARS-CoV-2 spike protein, and SV-40 transcription termination signal were inserted between the adenoviral genes for fiber and E4 ORF6/7. A schematic of the sVAX-19 genome is shown in FIG. 2. For Geist-20, the same expression cassette was used as for sVAX-19 and additionally an expression cassette with the ferritin light chain promoter driving cDNA encoding SARS-CoV-2 M protein with the beta actin transcription terminator and the ferritin heavy chain promoter driving cDNA encoding SARS-CoV-2 E protein with the RPS11 transcription terminator was cloned between the adenoviral IX and IVa2 genes, with a portion of the GAPDH transcription terminator to terminate transcription of the adenoviral IX gene. A schematic of the Geist-20 genome is shown in FIG. 3. Upon infection of host cells with Geist-20 in vivo, SARS-CoV-2 virus like particles (VLPs) are produced, which are contemplated to elicit both humoral and cellular immune response against the SARS-CoV proteins.

The expression cassette sequence in which the nucleotide sequence encoding SARS-CoV S was inserted within the adenovirus L5-E4 insertion site is:

(SEQ ID NO: 14)
tttcatacattgcccaagaataaAAGGTTTATTAGGCGGCCTCCCCGTCA
CCACCCCCCCCAACCCGCCCCGACCGGAGCTGAGAGTAATTCATACAAAA
GGACTCGCCCCTGCCTTGGGGAATCCCAGGGACCGTCGTTAAACTCCCAC
TAACGTAGAACCCAGAGATCGCTGCGTTCCCGCCCCCTCACCCGCCCGCT
CTCGTCATCACTGAGGTGGAGAAGAGCATGCGTGAGGCTCCGGTGCCCGT
CAGTGGGCAGAGCGCACATCGCCCACAGTCCCCGAGAAGTTGGGGGGAGG
GGTCGGCAATTGAACCGGTGCCTAGAGAAGGTGGCGCGGGGTAAACTGGG
AAAGTGATGTCGTGTACTGGCTCCGCCTTTTTCCCGAGGGTGGGGGAGAA
CCGTATATAAGTGCAGTAGTCGCCGTGAACGTTCTTTTTCGCAACGGGTT
TGCCGCCAGAACACAATTTAGAAACAAACCAACCTGTCTGTATTAGCCAC
CGCCACC*AAATAACTTGTTTATTGCAGCTTATAATGGTTACAaataaag
aatcgtttgtgttatgtttcaacg.

Sequence elements of the SARS-CoV S protein expression cassette in order include:

Lowercase: Flanking adenoviral sequence including part of fiber;
Underlined: Bidirectional transcription termination signal;
Plain: EF1A promoter;
Underlined: Partial SwaI restriction site;
Plain: Cumate operator;
Underlined: Kozak consensus sequence;
Asterisk: Insertion site for SARS-CoV S protein coding sequence;
Underlined: Partial SwaI restriction site;
Plain: SV-40 terminator; and
Lowercase: Flanking adenoviral sequence.

The expression cassette sequence in which the nucleotide sequence encoding SARS-CoV M and the nucleotide sequence encoding SARS-CoV E were inserted within the adenovirus IX-E2 insertion site is:

(SEQ ID NO: 20)
cccctcccaatgcggtttaaaacataaataaaGTACCCTGTGCTCAACCA
GTTACTTGTCCTGTCTTATTCTAGGGTCTGGGGCCCCAACCCAGCCACAC
CACAAAGTCACACTTGGCCTCATTTTTAAGGTGTGCACTTTTATTCAACT
GGTCTCAAGTCAGTGTACAGGTAA*GGTGGCTAATACAGACAGGTTGGTT
TGTTTCTCTGTTGAAGCAAGAGACAGACCCGCGGGACCGCCGAACTGCGA
GGGGACGTGGCTAGGGCGGCTTCTTTTATGGTGCGCCGGCCCTCGGAGGC
AGGGCGCTCGGGGAGGGCTAGCGGCCAATCTGCGGTGGCAGGAGGCGGGG
CCGAAGGCCGTGCCTGACCAATCCGGAGCACATAGGAGTCTCAGCCCCCC
GCCCCAAAGCAAGGGGAAGTCACGCGCCTGTAGCGCCAGCGTGTTGTGAA
ATGGGGGCTTGGGGGGGTTGGGGCCCTGTCCGCCAGAGCGCGCGAGGGCC
TCCAGCGGCCGCCCCTCCCCCACAGCAGGGGCGGGGTCCCGCGCCCACCG
GAAGGAGCGGGCTCGGGGCGGGCGGCGCTGATTGGCCGGGGCGGGCCTGA
CGCCGACGCGGCTATAAGAGACCACAAGCGACCCGCAGGGCCAGACGTTC
TTCGCCGAGAGAACAAACAGACAATCTGGTCTGTTTGTAGCCACC*GACA
TCGGCCCGCTCCCCACAATGAAATAAAaaaccagactctgtttggatttg
gatcaagcaagtgtcttgctgtctttatttaggggttttgcgcgcg.

Sequence elements of the SARS-CoV M and E protein expression cassette in order include:

Lowercase: Flanking adenoviral sequence including part of IX;
Underlined: Partial GAPDH terminator;
Plain: Beta actin terminator (reverse complement);
Asterisk: Insertion site for SARS-CoV M protein coding sequence (reverse complement);
Underlined: Kozak consensus sequence (reverse complement);
Plain: Cumate operator site (reverse complement);
Underlined: Ferritin light chain promoter (reverse complement);
Plain: Ferritin heavy chain promoter;
Underlined: Cumate operator site;
Plain: Kozak consensus sequence;
Asterisk: Insertion site for SARS-CoV E protein coding sequence;
Plain: RPS11 terminator; and
Lowercase: Flanking adenoviral sequence including part of IVa2.

The plasmids were digested with Pad and fused with a sequence independent ligation and cloning (SLIC) reaction, and transfected into SF-BMAdR cells to rescue the recombinant viruses. The viruses were amplified in SF-BMAdR cells. SF-BMAdR cells were derived from the A549, human epithelial cell line. The viruses are prepared as a pharmaceutical by purifying the adenoviruses from infected cells using procedures known in the field of adenovirus manufacturing, such as lysis with detergent, filtration, and chromatography in various combinations. The viruses are formulated as pharmaceutical compositions by suspension in sterile buffers (e.g., 25 mM NaCl, 20 mM Tris, 2.5% glycerol, etc.) known by those skilled in the art of adenovirus manufacture.

### Example 2. Active Immunization with a SARS-CoV-2 Adenoviral Vector

This example describes Phase 1 and Phase 2/3 clinical studies to assess safety, immunogenicity and efficacy of the recombinant human adenovirus type 5 (Ad5) vectors engineered to express SARS-CoV-2 antigens (COVID-19 vaccines), which are described in Example 1.

### Phase 1 Clinical Study

The Phase I study is conducted as an open-label, single arm, dose-escalation study in healthy adults, who are 18-55 years old and who have not been infected with or exposed to SARS-CoV-2. Infection and exposure are determined by nucleic acid and antibody tests, respectively. Subjects receive a single dose by subcutaneous injection of a COVID-19 vaccine. Each dose contains a low, medium or high number of recombinant Ad5 particles.

### Phase 2/3 Clinical Study—Close-Contact Design

The Phase 2/3 study is conducted as a randomized, blinded, placebo-controlled, two arm study in asymptomatic household contacts (study subjects) of SARS-CoV-2-infected patients. The study subjects are adults who are 18-55 years old and who do not exhibit COVID-19 symptoms. SARS-CoV-2 infection is determined by nucleic acid tests. Subjects receive a single dose by subcutaneous injection of a placebo formulation or a COVID-19 vaccine at a dose determined from the Phase I study.

### Phase 2/3 Clinical Study—Traditional Design

The Phase 2/3 study is conducted as a randomized, blinded, placebo-controlled, two arm study in healthy adults, who are 18 years of age or older and who have not been infected with or exposed to SARS-CoV-2. Infection and exposure are determined by nucleic acid and antibody tests, respectively. Subjects receive a single dose by subcutaneous injection of a placebo formulation or a COVID-19 vaccine at a dose determined from the Phase I study. Study subjects are stratified into three groups: 18-55 years, 56-70 years, and 71 years and older.

### Safety, Immunogenicity and Efficacy

The occurrence of adverse events, including vaccine-related and/or infection-related adverse events, in study subjects is monitored to assess vaccine safety. Blood samples are taken before administration (baseline) and periodically after administration (e.g., 3 months, 6 months, 12 months, etc.) of the COVID-19 vaccine to assess development of SARS-CoV-2-reactive humoral and cellular immune responses. In particular, SARS-CoV-2 neutralizing antibody titers are measured and the presence of SARS-CoV reactive CD4+ and CD8+ T cells is assessed. Additionally, ratios of virus-neutralizing antibody titers to virus-binding antibody titers are determined, and Th1 versus Th2 polarization is assessed. Study subjects are followed for about 1-2 years post-vaccination to evaluate risk of vaccine-enhanced disease and durability of immune responses. Outcome measures of the Phase 2/3 studies may include one or more of SARS-CoV-2 infection rates, COVID-19 disease development rates, and persistence of neutralizing antibody titers at one or more landmark dates (e.g., 12 months post-immunization).

### Example 3. Passive Immunization with IgG From Subjects Vaccinated with a SARS-CoV-2 Adenoviral Vector

This example describes an early phase clinical study to assess safety and efficacy of immune globulin obtained from healthy adult subjects immunized with COVID-19 vaccines as described in Examples 1 and 2.

SARS-CoV-2 immune globulin (SIG) is a composition comprising purified immunoglobulin derived from pooled plasma from healthy adult human subjects who were immunized with a COVID-19 vaccine. In brief, subjects who developed high titers of neutralizing antibodies against SARS-CoV-2 were selected for plasmapheresis. Plasma from the donors is pooled and fractionated, filtered and treated with solvent/detergent to inactive any residual virus that may be present. The immunoglobulin fraction may be further purified, lyophilized and reconstituted to obtain a sterile formulation suitable for intravenous injection. Some SARS-CoV-2 immune globulin formulations include primarily IgG, with trace amounts of IgA and IgM. Other SARS-CoV-2 immune globulin formulations include one or both of IgG2 and IgG4, but not one or both of IgG1 and IgG3. Enrichment of certain isotypes in the immune globulin is accomplished by either positive selection for the isotype(s) of interest, or negative selection against the isotype(s) of interest. Still other SARS-CoV-2 immune globulin formulations include only Fab or F(ab′)2 fragments.

The study is conducted in adults who are 18 years of age or older and who are hospitalized with COVID-19 respiratory symptoms and confirmed to be SARS-CoV-2-infected by nucleic acid testing. A SARS-CoV-2 immune globulin (SIG) formulation is administered by intravenous infusion on one or more occasions. The occurrence of adverse events, including SIG-related and/or infection-related adverse events, in study subjects is monitored to assess SIG safety. Outcome measures may include one or more of mortality rate (all cause) at one or more landmark dates (e.g., 28 days post-infusion), time to death, multi-organ failure progression, duration of hospitalization, supplemental oxygen-free days, ventilator-free days, intensive care unit (ICU)-free days, and SARS-CoV-2 viral load.

### Example 4. Treatment of Long COVID-19 by Active Immunization

This example describes clinical studies to assess safety, tolerability and efficacy of recombinant human adenovirus type 5 (Ad5) vectors engineered to express SARS-CoV-2 antigens (e.g., COVID-19 vaccines described in Example 1) administered to patients with long COVID.

### Phase 1 Clinical Study

The focus of the Phase 1 study is on assessing safety and tolerability, and determining the maximum tolerated dose (MTD) in a standard serial (3+3) cohort dose escalation. A dose expansion cohort follows MTD determination to establish the Phase 2 recommended dose (P2RD). The Phase I study is conducted in adults that were previously infected with SARS-CoV-2, who are 18 years of age or older. Prior infection is determined by nucleic acid and/or and antibody tests, respectively. Subjects receive either one dose (prime) or two doses (prime+boost) of a COVID-19 vaccine by intradermal injection. Dosages tested are in a range of from 1×10⁵ to 1×10¹⁰ recombinant Ad5 particles.

### Phase 2 Clinical Study

The Phase 2 study is conducted as a dose-ranging study to assess efficacy.

The Phase 2 study is conducted in adults with long COVID, who are 18 years of age or older. Long COVID, also referred to as post COVID syndrome, post-acute sequelae of COVID-19, chronic COVID syndrome and long haul COVID, is a condition that may result as a consequence of SARS-CoV-2 infection. Long COVID is characterized by symptoms of COVID-19 continuing or developing after acute SARS-CoV-2 infection (see, e.g., Amenta et al., Open Forum Infect Dis, 7(12): pfaa509. 2020). Inclusion criteria include a positive result for SARS-CoV-2 infection by reverse-transcriptase polymerase chain reaction testing within three or more weeks of enrollment, and one or more symptoms of COVID-19. Symptoms of COVID-19 include but are not limited to fatigue, headache, dyspnea (labored breathing), polypnea (rapid breathing), cognitive dysfunction, anosmia (loss of smell), ageusia (loss of taste), cough, joint pain, muscle pain, chest pressure, depression, anxiety and palpitations. Subjects receive one or two doses of a placebo formulation, or one dose (prime) or two doses (prime+boost) of a COVID-19 vaccine by intradermal injection. The dosage of the COVID-19 vaccine is determined from the Phase 1 study (P2RD) described above.

For the duration of the study (baseline on day 0 through day 56), subjects are asked to self-report severity of COVID-19 symptoms daily in a symptom diary using a severity score. Subjects are also evaluated using PROMIS® (Patient-Reported Outcomes Measurement Information System), which was developed through an initiative of the U.S. Department of Health and Human Services (Cella et al., “Patient-Reported Outcomes in Performance Measurement.” Research Triangle Park (NC), RTI Press, 2015). PROMIS® is a set of measures that evaluate and monitor physical, mental, and social health in adults and children, including individuals living with chronic conditions such as long COVID. Through a series of guided questions over short form or computer, PROMIS® measures are scored on the T-score metric such that 50 is the mean of the reference population and 10 is the standard deviation of that same population. For example, a score of 40 is one standard deviation lower than the mean for the reference population. Therefore, higher sores correlate with more of the metric being measured (i.e., more fatigue, more pain, etc.).

The primary efficacy endpoint is a change from baseline on day 0 through day 56 in the daily COVID-19 related symptom severity score (e.g., reduction in severity score). Secondary endpoints based on self-assessment using the daily symptom diary include one or both of: 1) Duration of COVID-19 associated symptoms from baseline on day 0; and 2) Number of symptom-free days of COVID-19 associated symptoms that were present on baseline on day 0. Additional secondary endpoints may include one or more of: 3) Progression (or worsening) of COVID-19-associated symptoms through Day 56 compared to baseline on Day 0; 4) Change from baseline in PROMIS Fatigue Score at Day 28 and Day 56; 5) Change from baseline in PROMIS Cognitive Function Score at Day 28 and Day 56; 6) Duration (days) of hospitalization from baseline on Day 0 through Day 56; and 7) Incidence of hospitalization from baseline on Day 0 through Day 56.

### Phase 3 Clinical Study

The Phase 3 study is conducted as a randomized, blinded, placebo-controlled, two arm study in adults with long COVID, who are 18 years of age or older. Inclusion criteria include a positive result for SARS-CoV-2 infection by reverse-transcriptase polymerase chain reaction testing within three or more weeks of enrollment, and one or more symptoms of COVID-19. Symptoms of COVID-19 include but are not limited to fatigue, headache, dyspnea (labored breathing), polypnea (rapid breathing), cognitive dysfunction, anosmia (loss of smell), ageusia (loss of taste), cough, joint pain, muscle pain, chest pressure, depression, anxiety and palpitations. Subjects receive one or two doses of a placebo formulation, or one dose (prime) or two doses (prime+boost) of a COVID-19 vaccine by intradermal injection and who have been infected with or exposed to SARS-CoV-2 and have received a COVID-19 diagnosis. The dosage of the COVID-19 vaccine is determined from the Phase 1 study (P2RD) described above.

Measurement and evaluation of primary and secondary endpoints is as described for the Phase 2 study. The analysis of the primary efficacy endpoints is to use a Wilcoxon rank sum test (two sided at the 0.05 significance level). A general linear model analysis of covariance is to use a sensitivity analysis for the Wilcoxon test.

### Example 5. Treatment of Cancer by Active Immunization

This example describes clinical studies to assess safety, tolerability and efficacy of the recombinant human adenovirus type 5 (Ad5) vectors engineered to express SARS-CoV-2 antigens (e.g., COVID-19 vaccines described in Example 1) administered to patients with cancer. The rationale of the clinical trials of this example are based in part on anecdotal reports of cancer remission after SARS-CoV-2 infection (Sollini et al., Eur J Nucl Med Mol Imaging, 2021; 1-3, Feb. 2021), which is contemplated to be related to the occurrence of a coronavirus-induced cytokine storm and/or other characteristics shared by coronaviruses. In particular, the coronavirus nucleocapsid (N) protein has cell cycle inhibitory activity through direct binding to cyclin D or cyclin CDK-2, and indirect downregulation of other cyclins (Su et al., Front Vet Sci, 7:586826, 2020).

### Phase 1 Clinical Study

The focus of the Phase 1 study is on assessing safety and tolerability, and determining the maximum tolerated dose (MTD) in a standard serial (3+3) cohort dose escalation. A dose expansion cohort follows MTD determination to establish the Phase 2 recommended dose (P2RD). The Phase I study is conducted in adults with cancer, who are 18 years of age or older. Subjects receive either one dose (prime) or two doses (prime+boost) of a COVID-19 vaccine by intradermal or intravenous injection. Dosages tested are in a range of from 1×10⁹ to 1×10¹² recombinant Ad5 particles.

### Phase 2 Clinical Study

The Phase 2 study is conducted as a basket design, dose-ranging study to assess efficacy. The Phase 2 study is conducted in adults with cancer (solid tumor), who are 18 years or older. Subjects receive one or two doses of a placebo formulation, or one dose (prime) or two doses (prime+boost) of a COVID-19 vaccine by intradermal, intratumoral or intravenous injection. A delivery vehicle (e.g., nanoparticles or liposomes) may be used. The dosage of the COVID-19 vaccine is determined from the Phase 1 study (P2RD) described above.

Patients are evaluated using guidelines of the Response Evaluation Criteria in Solid Tumors (RECIST version 1.1) to assess efficacy of the COVID-19 vaccines for treating cancer. RECIST guidelines are as described (see, e.g., Eisenhauer et al., Eur J Cancer, 45:228-247, 2009; and Nishino et al., Am J Roentgenol, 195: 281-289, 2010). Response criteria to determine objective anti-tumor responses per RECIST 1.1 include: complete response (CR); partial response (PR); progressive disease (PD); and stable disease (SD). The RECIST overall objective response (ORR) is estimated with its corresponding Clopper-Pearson at 95% confidence interval (CI). Progression free survival (PFS) and overall survival (OS) is estimated using the Kaplan-Meier method. Median survival is derived using the Brookmeyer-Crowley method at 95% CI.

Additionally or alternatively, patients are evaluated using guidelines of the Immunotherapy Response Evaluation Criteria in Solid Tumors (iRECIST) as described (see, e.g., Seymour et al., Lancet Oncol, 18(3):e143-e152, 2017). A significant difference between RECIST 1.1 and iRECIST is that iRECIST resets the bar when RECIST 1.1 progression is followed by tumor shrinkage. The Response criteria to determine objective anti-tumor responses per iRECIST include: immune complete response (iCR); immune complete progression (iCPD); immune partial response (iPR); immune stable disease (iSD); and immune unconfirmed progression (iUPD).

### Phase 3 Clinical Study

The Phase 3 study is conducted as a randomized, blinded, placebo-controlled, two arm study to assess efficacy. The Phase 3 study is conducted in adults with cancer (solid tumor), who are 18 years of age or older. Subjects receive one or two doses of a placebo formulation, or one dose (prime) or two doses (prime+boost) of a COVID-19 vaccine by intradermal, intratumoral or intravenous injection. A delivery vehicle (e.g., nanoparticles or liposomes) may be used. The dosage of the COVID-19 vaccine is determined from the Phase 1 study (P2RD) described above.

Efficacy is assessed as described for the Phase 2 study. The primary endpoint is either overall survival (OS) or progression free survival (PFS). Choice or primary endpoint is dependent on the specific cancer type and previous treatments of the study subjects. A log-rank test is used to compare survival curves. A Cox model is employed to derive the hazard ratio estimate and its 95% CI to ascertain the magnitude of the observed effect size.

1: A recombinant human adenovirus for stimulating an immune response against a coronavirus in a human subject, wherein
the adenovirus comprises a nucleotide sequence encoding a structural protein of a coronavirus located at an insertion site in the adenovirus genome,
the coronavirus structural protein comprises a nucleocapsid protein, a spike protein, a membrane protein, an envelope protein, a fragment thereof, or a combination thereof, and
the adenovirus genome comprises a modified E1a transcription regulatory sequence.

2: The recombinant human adenovirus of claim 1, wherein the recombinant adenovirus is a type 5 adenovirus (Ad5).

3: The recombinant human adenovirus of claim 2, wherein the coronavirus is an alphacoronavirus, a betacoronavirus, a gammacoronavirus, or a deltacoronavirus, optionally wherein the coronavirus is gammacoronavirus selected from the group consisting of a severe acute respiratory syndrome coronavirus 2 (SARS-CoV-2), a severe acute respiratory syndrome coronavirus 1 (SARS-CoV-1), and a middle east respiratory syndrome-related coronavirus (MERS-CoV), optionally wherein the coronavirus is a severe acute respiratory syndrome coronavirus 2 (SARS-CoV-2).

4: The recombinant human adenovirus of claim 3, wherein the insertion site is selected from the group consisting of an E1b-19K insertion site, an E3 insertion site, an E4 insertion site, an IX-E2 insertion site, an L5-E4 insertion site, and combinations thereof.

5: The recombinant human adenovirus of claim 4, wherein the modified E1a transcription regulatory sequence is a modified E1a promoter, optionally the modified E1a promoter comprises a deletion of nucleotides corresponding to 195-244 of the Ad5 genome (SEQ ID NO:1).

6-7. (canceled)

8: The recombinant human adenovirus of claim 3, wherein the coronavirus structural protein comprises a coronavirus nucleocapsid protein or a fragment thereof.

9: The recombinant human adenovirus of claim 8, wherein the nucleotide sequence encoding the nucleocapsid protein or a fragment thereof is located at the E1b-19K insertion site between the start site of E1b-19K and the stop codon of E1b-19K in place of about 200 nucleotides of E1b-19K, optionally the E1b-19K insertion site is between nucleotides corresponding to 1714 and 1916 of the Ad5 genome (SEQ ID NO:1).

10-11. (canceled)

12: The recombinant human adenovirus of claim 3, wherein the adenovirus genome comprises a deletion of at least a portion of E3.

13: The recombinant human adenovirus of claim 12, wherein the coronavirus structural protein comprises a coronavirus spike protein or a fragment thereof.

14: The recombinant human adenovirus of claim 13, wherein the nucleotide sequence encoding the spike protein or fragment thereof is located at the L5-E4 insertion site between the stop codon of adenovirus fiber gene and the stop codon of adenovirus E4-ORF6/7 gene.

15: The recombinant human adenovirus of claim 14, wherein the L5-E4 insertion site is between nucleotides corresponding to 32787 and 32914 of the Ad5 genome (SEQ ID NO:1) or the nucleotide sequence encoding the spike protein or a fragment thereof is contained within an expression cassette comprising residues 24-543 of the sequence of SEQ ID NO:14.

16. (canceled)

17: The recombinant human adenovirus of claim 3, wherein the coronavirus structural protein comprises a coronavirus membrane protein or a fragment thereof, optionally wherein the nucleotide sequence encoding the membrane protein or fragment thereof is contained within an expression cassette comprising residues 33-478 of the sequence of SEQ ID NO:20, optionally wherein the nucleotide sequence encoding the membrane protein or fragment thereof is located at the IX-E2 insertion site between the stop codon of adenovirus IX gene and the stop codon of adenovirus IVa2 gene, further optionally wherein the IX-E2 insertion site is between nucleotides corresponding to 4029 and 4093 of the Ad5 genome (SEQ ID NO:1).

18. (canceled)

19: The recombinant human adenovirus of claim 3, wherein the coronavirus structural protein comprises a coronavirus envelope protein or a fragment thereof, optionally wherein the nucleotide sequence encoding the envelope protein or a fragment thereof is contained within an expression cassette comprising residues 479-727 of the sequence of SEQ ID NO:20, optionally wherein the nucleotide sequence encoding the envelope protein or fragment thereof is located at the IX-E2 insertion site between the stop codon of adenovirus IX gene and the stop codon of adenovirus IVa2 gene, further optionally wherein the IX-E2 insertion site is between nucleotides corresponding to 4029 and 4093 of the Ad5 genome (SEQ ID NO:1).

20. (canceled)

21: The recombinant human adenovirus of claim 3, wherein the coronavirus structural protein comprises a coronavirus membrane protein or a fragment thereof and a coronavirus envelope protein or a fragment thereof, optionally wherein the nucleotide sequence encoding the envelope protein or a fragment thereof and the nucleotide sequence encoding the membrane protein or a fragment thereof are contained within an expression cassette comprising residues 33-727 of the sequence of SEQ ID NO:20.

22: The recombinant human adenovirus of claim 3, wherein the adenovirus genome comprises a deletion of at least a portion of E4 and/or a deletion of at least a portion of E3.

23: A kit comprising:
i) the recombinant human adenovirus of claim 1, and
ii) instructions for administration of the adenovirus to stimulate an immune response against the coronavirus structural antigen in the human subject, optionally further comprising a syringe and needle for injection of the recombinant adenovirus, optionally by intramuscular, subcutaneous, intradermal, intratumoral or intravenous injection, optionally by intramuscular or subcutaneous injection.

24. (canceled)

25: A method for (i) stimulating an immune response against a coronavirus in a human subject, (ii) treating long COVID-19 in a human subject, or (iii) treating cancer in a human subject, comprising administering an effective amount of the recombinant human adenovirus of claim 1 to a human subject.

26-28. (canceled)

29: A method for preparing a coronavirus immune globulin preparation, the method comprising:
(a) immunizing a plurality of healthy adult human subjects between the ages of 18-60 with an effective amount of the recombinant human adenovirus of claim 1 to elicit coronavirus neutralizing antibodies;
(b) harvesting plasma from the immunized subjects, optionally wherein the plasma is harvested by plasmapheresis;
(c) pooling the plasma to obtain a pooled plasma preparation comprising the coronavirus neutralizing antibodies; and
(d) fractionating the pooled plasma preparation to obtain coronavirus immune globulin preparation.

30-33. (canceled)

34: A coronavirus immune globulin preparation prepared according to the method of claim 29.


SEQUENCE LISTING (34 sequences)

SEQ ID NO: 1 - DNA, 32306 bp, Adenovirus type 5
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcac

SEQ ID NO: 2 - DNA, 10 bp, Artificial Sequence
  Synthetic Construct
ggtgttttgg

SEQ ID NO: 3 - DNA, 10 bp, Artificial Sequence
  Synthetic Construct
gactgtgcgc

SEQ ID NO: 4 - DNA, 8 bp, Artificial Sequence
  Synthetic Construct
ctgacctc

SEQ ID NO: 5 - DNA, 8 bp, Artificial Sequence
  Synthetic Construct
tcaccagg

SEQ ID NO: 6 - protein, 3 aa, Artificial Sequence
  Synthetic Construct
AAY

SEQ ID NO: 7 - DNA, 46 bp, Artificial Sequence
  Synthetic Construct
atcttggttacatctgacctcgtcgagtcaccaggcgcttttccaa

SEQ ID NO: 8 - DNA, 8 bp, Artificial Sequence
  Synthetic Construct
cagtatga

SEQ ID NO: 9 - DNA, 10 bp, Artificial Sequence
  Synthetic Construct
taataaaaaa

SEQ ID NO: 10 - DNA, 10 bp, Artificial Sequence
  Synthetic Construct
ttccgtggcg

SEQ ID NO: 11 - DNA, 8 bp, Artificial Sequence
  Synthetic Construct
tgccttaa

SEQ ID NO: 12 - DNA, 11 bp, Artificial Sequence
  Synthetic Construct
taaaaaaaaat

SEQ ID NO: 13 - DNA, 553 bp, Artificial Sequence
  Synthetic Construct
taacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 14 - DNA, 573 bp, Artificial Sequence
  Synthetic Construct
tttcatacattgcccaagaataaaaggtttattaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaatttagaaacaaaccaacctgtctgtattagccaccgccaccaaataacttgtttattgcagcttataatggttacaaataaagaatcgtttgtgttatgtttcaacg

SEQ ID NO: 15 - DNA, 8 bp, Artificial Sequence
  Synthetic Construct
tattcccg

SEQ ID NO: 16 - DNA, 8 bp, Artificial Sequence
  Synthetic Construct
agtgcccg

SEQ ID NO: 17 - DNA, 9 bp, Artificial Sequence
  Synthetic Construct
ctaggactg

SEQ ID NO: 18 - protein, 4 aa, Artificial Sequence
  Synthetic Construct
  VARIANT 2: Xaa = Any Amino Acid
  VARIANT 3: Xaa = Lys or Arg
RXXR

SEQ ID NO: 19 - protein, 4 aa, Artificial Sequence
  Synthetic Construct
RAKR

SEQ ID NO: 20 - DNA, 794 bp, Artificial Sequence
  Synthetic Construct
cccctcccaatgcggtttaaaacataaataaagtaccctgtgctcaaccagttacttgtcctgtcttattctagggtctggggccccaacccagccacaccacaaagtcacacttggcctcatttttaaggtgtgcacttttattcaactggtctcaagtcagtgtacaggtaaggtggctaatacagacaggttggtttgtttctctgttgaagcaagagacagacccgcgggaccgccgaactgcgaggggacgtggctagggcggcttcttttatggtgcgccggccctcggaggcagggcgctcggggagggctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgtccgccagagcgcgcgagggcctccagcggccgcccctcccccacagcaggggcggggtcccgcgcccaccggaaggagcgggctcggggcgggcggcgctgattggccggggcgggcctgacgccgacgcggctataagagaccacaagcgacccgcagggccagacgttcttcgccgagagaacaaacagacaatctggtctgtttgtagccaccgacatcggcccgctccccacaatgaaataaaaaaccagactctgtttggatttggatcaagcaagtgtcttgctgtctttatttaggggttttgcgcgcg

SEQ ID NO: 21 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 22 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - protein, 73 aa, SARS-CoV-2
NSNNLDSKVGGNYNYLYRLFRKSNLKPFERDISTEIYQAGSTPCNGVEGFNCYFPLQSYGFQPTNGVGYQPYR

SEQ ID NO: 24 - protein, 222 aa, SARS-CoV-2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 25 - protein, 75 aa, SARS-CoV-2
MYSFVSEETGTLIVNSVLLFLAFVVFLLVTLAILTALRLCAYCCNIVNVSLVKPSFYVYSRVKNLNSSRVPDLLV

SEQ ID NO: 26 - protein, 422 aa, SARS-CoV-1
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 27 - protein, 1255 aa, SARS-CoV-1
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 28 - protein, 221 aa, SARS-CoV-1
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 29 - protein, 76 aa, SARS-CoV-1
MYSFVSEETGTLIVNSVLLFLAFVVFLLVTLAILTALRLCAYCCNIVNVSLVKPTVYVYSRVKNLNSSEGVPDLLV

SEQ ID NO: 30 - protein, 411 aa, MERS-CoV [sequence omitted]

SEQ ID NO: 31 - protein, 1353 aa, MERS-CoV
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRFLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 32 - protein, 219 aa, MERS-CoV
MSNMTQLTEAQIIAIIKDWNFAWSLIFLLITIVLQYGYPSRSMTVYVFKMFVLWLLWPSSMALSIFSAVYPIDLASQIISGIVAAVSAMMWISYFVQSIRLFMRTGSWWSFNPETNCLLNVPFGGTTVVRPLVEDSTSVTAVVTNGHLKMAGMHFGACDYDRLPNEVTVAKPNVLIALKMVKRQSYGTNSGVAIYHRYKAGNYRSPPITADIELALLRA

SEQ ID NO: 33 - protein, 82 aa, MERS-CoV
MLPFVQERIGLFIVNFFIFTVVCAITLLVCMAFLTATRLCVQCMTGFNTLLVQPALYLYNTGRSVYVKFQDSKPPLPPDEWV

SEQ ID NO: 34 - DNA, 9 bp, Artificial Sequence
  Synthetic Construct
  misc_feature 3: n = T or C
ggncaatct